(19) United States
(12) Patent Application Publication
(10) Pub. No.: US 2025/0108141 A1
(43) Pub. Date: Apr. 3, 2025

(54) TOPICAL FORMULATIONS FOR TARGETED DELIVERY OF THERAPEUTICS

(71) Applicant: UNM RAINFOREST INNOVATIONS, Albuquerque, NM (US)

(72) Inventors: Jason Thomas McConville, Albuquerque, NM (US); Michelle A. Ozbun, Albuquerque, NM (US)

(21) Appl. No.: 18/899,983

(22) Filed: Sep. 27, 2024

Related U.S. Application Data
(60) Provisional application No. 63/541,323, filed on Sep. 29, 2023.

(51) Int. Cl. A61L 15/44; A61K 31/444; A61K 31/4523; A61K 31/519; A61K 47/38; A61P 31/20

(52) U.S. Cl. CPC A61L 15/44; A61K 31/444; A61K 31/4523; A61K 31/519; A61K 47/38; A61P 31/20; A61L 2300/408

(57) ABSTRACT

Pharmaceutical compositions including a gelation agent for topical delivery of active agents are disclosed. Methods for treatment of epithelial disorders using topical application of the pharmaceutical compositions disclosed herein are also described. A kit including the pharmaceutical compositions disclosed herein is also described.

### CROSS REFERENCE TO RELATED APPLICATIONS

This application claims the benefit of 63/541,323, filed Sep. 29, 2023, the disclosure of which is incorporated by reference herein in its entirety.

### GOVERNMENT FUNDING

This invention was made with government support under DE024305, CA118100, and DE028652 awarded by the National Institutes of Health. The government has certain rights in the invention.

### SUMMARY

In one aspect, this disclosure describes a pharmaceutical composition. Generally, the pharmaceutical composition includes an inhibitor of a protein implicated in viral replication, a gelation agent, and an aqueous solvent.

In one or more embodiments, the gelation agent includes hydroxypropyl methylcellulose, methylcellulose, or hydroxypropyl cellulose.

In one or more embodiments, the gelation agent has a molecular weight of 10 kDa to 2,000 kDa.

In one or more embodiments, the protein kinase inhibitor is an MEK inhibitor or an ERK inhibitor.

In one or more embodiments, the aqueous solvent includes water, alcohol, or a combination thereof. In one or more of these embodiments, the alcohol includes 2-(2-ethoxyethoxy)ethanol.

In one or more embodiments, the aqueous solvent is at least 40% of the pharmaceutical composition by weight. In one or more of these embodiments, the aqueous solvent is at least 80% of the pharmaceutical composition by weight.

In one or more embodiments, the pharmaceutical composition comprises at most 5% organic solvent by weight.

In one or more embodiments, the gelation agent forms a mucoadhesive hydrogel upon contact with a mucous membrane.

In one or more embodiments, the composition forms a gel before it is applied to a treatment site.

In another aspect, this disclosure describes a drug delivery device. Generally, the drug delivery device includes a pharmaceutical composition that includes a protein kinase inhibitor, a gelation agent, and an aqueous solvent.

In one or more embodiments, the drug delivery device includes a transdermal drug delivery device or topical drug delivery device.

In one or more embodiments, the drug delivery device includes a bandage, a wrap, a plaster, or a dressing.

In another aspect, this disclosure describes a container that includes a pharmaceutical composition that includes a protein kinase inhibitor, a gelation agent, and an aqueous solvent; and an applicator.

In another aspect, this disclosure describes a kit that includes a pharmaceutical composition that includes a protein kinase inhibitor, a gelation agent, and an aqueous solvent.

In another aspect, this disclosure describes a method of treating an epithelial disorder in a subject. Generally, the method includes applying a pharmaceutically active amount of a pharmaceutical composition to a treatment site. The pharmaceutical composition includes a protein kinase inhibitor, a gelation agent, and an aqueous solvent.

In one or more embodiments, the epithelial disorder is a viral disease, and wherein treating the subject results in a decrease of viral gene transcription relative to an untreated subject.

In one or more embodiments, the epithelial disorder is cancer, and wherein treating the subjects results in a decrease in tumor volume relative to an untreated subject.

In one or more embodiments, the treatment site is on a mucous membrane or an epidermal site.

In one or more embodiments, treating the subject results in a blood level of the protein kinase inhibitor of less than 100 ng/mL.

In one or more embodiments, treating the subject results in drug release at least 72 hours after application of the pharmaceutical composition.

In another aspect, this disclosure describes a method of preparing a pharmaceutical composition. Generally, the method includes preparing a solution that includes combining a gelation agent with an inhibitor of a protein kinase or a transcription activator and drying the solution to produce a dried composition. The protein kinase or transcription activator is a member of a cascade that regulates early viral gene expression or viral genome replication.

In one or more embodiments, the solution is dried by spray drying or freeze drying.

In one or more embodiments, the method further includes rehydrating the dried composition.

In one or more embodiments, the method further includes administering the dried composition to a treatment site.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

The patent or application file contains at least one photograph executed in color. Copies of this patent or patent application publication with color photographs will be provided by the Office upon request and payment of the necessary fee.

FIG. 1. Illustration comparing uninfected epithelium versus papilloma virus (PV) infected epithelium. Uninfected epithelium harbors few proliferating cells in the basal layer; as cells are pushed upward, they begin terminal differentiation to eventually form a cornified dead cell barrier. In PV-infected epithelium, expression of E6 and E7 proteins increases the proliferative compartment leading to hyper-proliferative diseases (warts, papillomas, condylomas, tumors). Note that p-ERK1/2 levels correlate with cell proliferative markers and, in HPV-infections, also with E7 protein activity (e.g., expression of p16INK4A, a surrogate of E7 activity).

FIG. 2. Histology data showing that HPV16 oncogene transcription and activity is modulated by MEK1/2 activation in 3D organotypic epithelial tissue cultures. HPV16-infected cells established from cervix (W12-E) were cultured as raft tissues for 14 days. Panels A-D are taken from tissues cultured under normal conditions. Panels E-H are images of tissue cultured with 10 ng/mL EGF added systemically (in the cell culture medium below the basal layer and collagen matrix) every 48 hours on days 8, 10, and 12 post raft tissue lifting to the air-liquid interface. Panels I-L are images of tissues cultured with 10 nM trametinib added systemically every 48-hours on days 8, 10, and 12 post raft tissue lifting to the air-liquid interface. Tissues were examined for morphology (Top, H&E staining), phosphorylated ERK1/2 (p-ERK1/2) immunohistochemistry (brown), HPV E6&E7 mRNA in situ hybridization (red), and E7 activity via proxy p16INK4A immunohistochemistry. Broken lines underscore the basal cell layer above the collagen matrix; bars=100 μm.

FIG. 3. Data showing that orally administered cobimetinib suppresses MmuPV1-induced benign tumor (e.g., wart) growth and trametinib leads to tumor regression on mouse tails. Female Hsd:Athymic Nude-Foxn1^{nu} mice were infected with 2×10⁸ viral genome equivalents (VGE) of MmuPV1 virions. Tumors (warts) were allowed to grow for 6 weeks before randomizing into vehicle or treatment groups (n=6 per group) with each group having an average tumor volume (length×width×height) of approximately 40 mm³. Mice were dosed by oral gavage treatment every 48 hours for four weeks before harvesting each tumor for histology. (A) Quantification of tail tumor volumes over 32 days of treatment in mice infected with MmuPV1 and treated with vehicle, 7.5 mg/kg cobimetinib, or 15 mg/kg cobimetinib. (B) Quantification of tail tumor volume over 32 days of treatment in mice infected with MmuPV1 and treated with vehicle (same as in FIG. 3A), 1.0 mg/kg trametinib, or 2.0 mg/kg trametinib. Statistical analyses by two-way Anova, Dunnett's T3 test (*p<0.5, ** p<0.01). (C) Representative images of tail tumors of a mouse treated with vehicle only. (D) Representative images of tail tumors of a mouse treated with 7.5 mg/kg cobimetinib or 15 mg/kg cobimetinib. (E) Representative images of tail tumors of a mouse treated with 1.0 mg/kg trametinib or 2.0 mg/kg trametinib.

FIG. 4. Data from tail tissue sections taken from mice infected with MmuPV1 and treated with orally administered formulations of vehicle, 15 mg/kg cobimetinib, or 2.0 mg/kg trametinib. Treated mice were female Hsd:Athymic Nude-Foxn1^{nu} mice; n=6/group. Broken lines underscore the basal cell layer. (A) First column; overall morphology of tails from vehicle-treated, cobimetinib-treated, or trametinib-treated mice stained with hematoxylin and eosin (H&E); Second column: p-ERK1/2 levels by visualized by immunohistochemistry (brown); Third column: expression of cytokeratin 10 visualized by immunohistochemistry; Fourth column: MmuPV1 viral genomes visualized by in situ hybridization (red); Fifth column: proliferative cytokeratin 14 positive cells (red) and MmuPV1 major capsid protein L1 positive foci (green) as visualized by immunofluorescence. (B) Quantification of each phenotype of interest: epithelial thickness, p-ERK1/2 abundance, cytokeratin 10, MmuPV1 DNA, or L1/cytokeratin 14 expression for five digital areas per tail (vehicle, 15 mg/kg cobimetinib, or 2.0 mg/kg trametinib). Statistical analyses by one-way Welch's Anova with Dunnett's T3 multiple comparison test (*p<0.05). Each histogram corresponds to the images presented above it.

FIG. 5. Diagram of an example experimental setup to test the effect of topical treatment with an MEK inhibitor on 3D organotypic epithelial raft tissues infected with HPV.

FIG. 6. Data demonstrating that topical trametinib treatment of HPV16 infected raft epithelial tissues suppresses p-ERK1/2 & HPV E6/E7 mRNA expression. HPV16-infected (SG3) cells were cultured as raft tissues for 12 days. Duplicate raft tissues were left untreated, or topically treated with 0.5 ml of either vehicle gel (20% Tc, 2.5% HPC in PBS) alone or 30 μM trametinib in vehicle gel for 48 hours. One raft from each duplicate set was collected for total protein extraction for SDS-PAGE and immunoblots (A), the second raft was harvested for tissue fixation, paraffin embedding, and cross-sectioning of tissues for histology (B-J). (A) Immunoblot data demonstrating an increase in p-ERK1/2 protein levels following vehicle only treatment of HPV16-infected raft tissues (lanes 3-4) compared to untreated raft tissues (lanes 1-2). HPV16-infected tissues treated topically with 30 μM trametinib in the vehicle showed a reduction in p-ERK1/2 to almost undetectable levels (lanes 5-6 and panel G). Panels B-D show histology by H&E staining in untreated, vehicle-treated, and 30 μM trametinib-treated rafts. Panels E-G show images of p-ERK1/2 immunohistochemistry in treated and control cells (brown) in agreement with immunoblot data from (A) where vehicle only increases p-ERK1/2 and trametinib abrogates p-ERK1/2 detection. Panels H-J show images of HPV16 E6/E7 mRNA in situ hybridization (red) in treated and control cells (with insets showing higher magnifications of the basal cells). Like the p-ERK1/2 results, vehicle only increases E6/E7 mRNA and trametinib reduces E6/E7 mRNA to undetectable levels. Broken lines underscore the basal cell layer.

FIG. 7. Immunoblot data from HPV16 infected epithelial raft tissues treated for 48 hours after applying a single treatment with formulations consistent with those described in this disclosure. HPC or vehicle only stimulates p-ERK1/2 and concomitantly E7 protein levels, whereas vehicle plus trametinib suppresses both p-ERK1/2 and HPV16 E7 protein expression.

FIG. 8. Matched histology data from epithelial raft tissues treated with formulations matched with those in FIG. 7 and consistent with those described in this disclosure. Labels B-H indicate the formulation used to treat the tissues shown, as is described in Example 2. In each case, adding trametinib to the formulation abrogated p-ERK1/2 protein and E6/E7 mRNA detection. Bottom panels include insets of higher magnification of the basal cells to better visualize E6/E7 mRNA detection (B, C, E, G) or lack thereof (D, F, H).

FIG. 9. Immunoblotting data from two replicates of the protocol described in Example 3 where HPV16 infected epithelial raft tissues treated for 72 hours after applying a single treatment. Lanes correspond to protein extracted from tissue treated with formulations A through H from left to right.

FIG. 10. Immunoblotting data of protein extracted from tissue treated with formulations B through H of Example 4.

FIG. 11. Immunoblotting data of protein extracted from tissue treated with MK-8353 in Example 5.

FIG. 12. Topical application of MEK inhibitor promotes MmuPV1 tumor regression in vivo. Mice bearing MmuPV1-induced tumors ranging in volume from 145.3 mm³ to 279.4 mm³ were treated daily with a topical gel (2.5% HPC, 15%-18% TRANSCUTOL (Gattefosse SAS, Saint-Priest, France)) containing the MEK inhibitor trametinib (150 μM-600 μM).

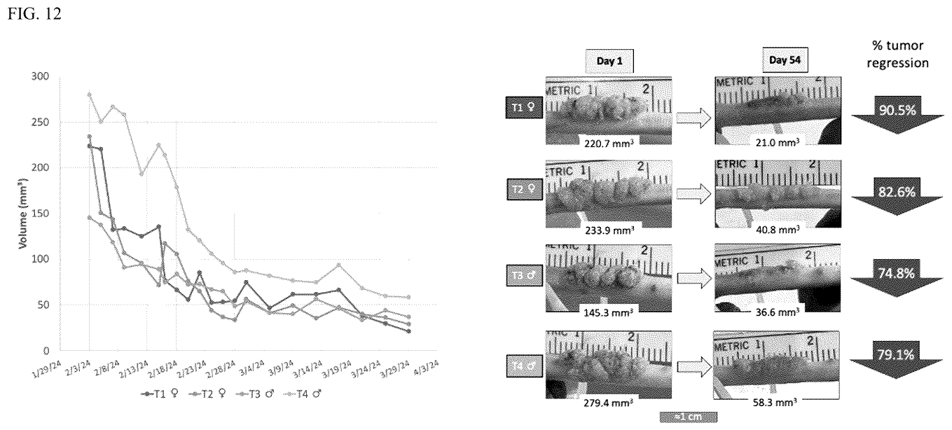

FIG. 13. Spray dispersion radius and impact droplet size of thermally gelling HPMC solutions. (A) 5% w/v HPMC E50; (B) 5% w/v HPMC E50+15% w/v DGME (Tc); (C) 2% w/v HPMC E50; (D) 2% w/v HPMC E50+15% w/v DGME; (E) 2% w/v HPMC E6; (F) 2% w/v HPMC E6+15% w/v DGME; (G) 1% w/v HPMC E50; (H) 1% w/v HPMC E50+15% w/v DGME.

FIG. 14. Sol-Gel transition or Tgel shown where G′ and G″ cross, demonstrating a tan δ value of 1.

FIG. 15. Sol-Gel transition or Tgel shown where G′ and G″ cross, demonstrating a tan δ value of 1.

FIG. 16. The application of 2% w/v HPMC E6+DGME with: (A) 1.5% NaF, and (B) 2.0% NaF by spraying onto a 37° C. hot plate from a distance of 7.9 cm.

FIG. 17. Sol-Gel transition shown where G′ (blue) and G″ (orange) cross, demonstrating a tan δ value of 1.

FIG. 18. Experiment akin to that in FIG. 6, except that it uses 2.5% HPC and 10% Tc for the vehicle. Drug treatment includes 15 μM trametinib. Experimental groups were treated with the topical formulations for 24 hours or 48 hours before harvesting for immunoblots and tissue sectioning. The vehicle formulations appear to activate p-ERK1/2 less than those used in FIG. 6. In each case, adding trametinib to the formulation reduced p-ERK1/2 protein and E6/E7 mRNA detection and 48-hour treatments were as effective as, if not more effective than, 24-hour treatments.

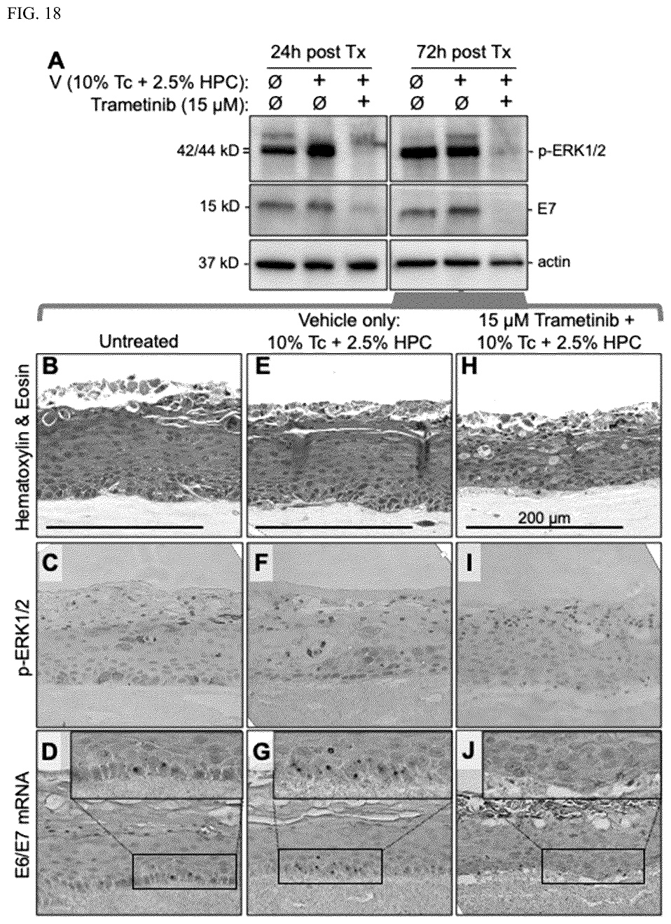

### DETAILED DESCRIPTION OF ILLUSTRATIVE EMBODIMENTS

This disclosure describes compositions and methods that may be used to treat proliferative or inflammatory skin diseases or disorders or skin diseases caused by infectious agents. In one aspect, the composition includes a formulation particularly suited for topical administration.

Dysregulation of cell signaling cascades in epithelial cells is a contributing factor to a wide range of skin and mucosal membrane disorders, including inflammatory skin disorders (e.g., psoriasis) and virally induced disorders (e.g., disorders related to papilloma virus infections). Papilloma viruses are a highly transmissible causative agents of benign and malignant tumors in mucosal and cutaneous squamous epithelium. Effective antiviral HPV therapy options are extremely limited.

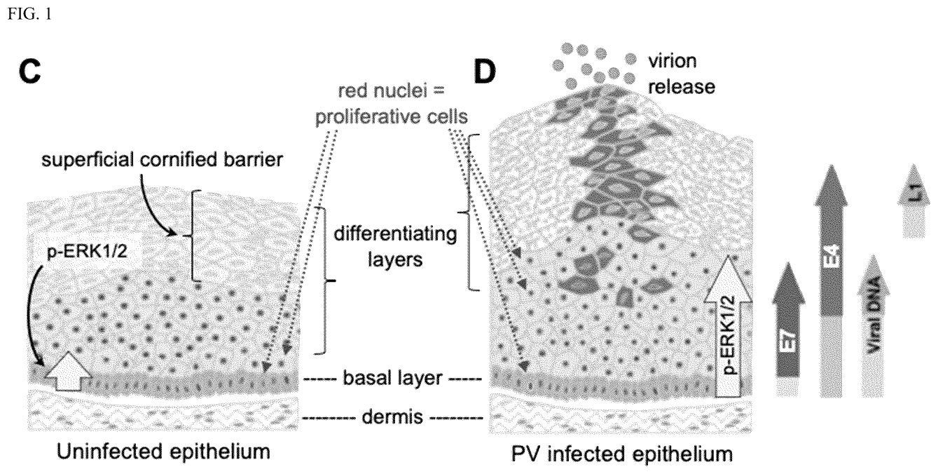

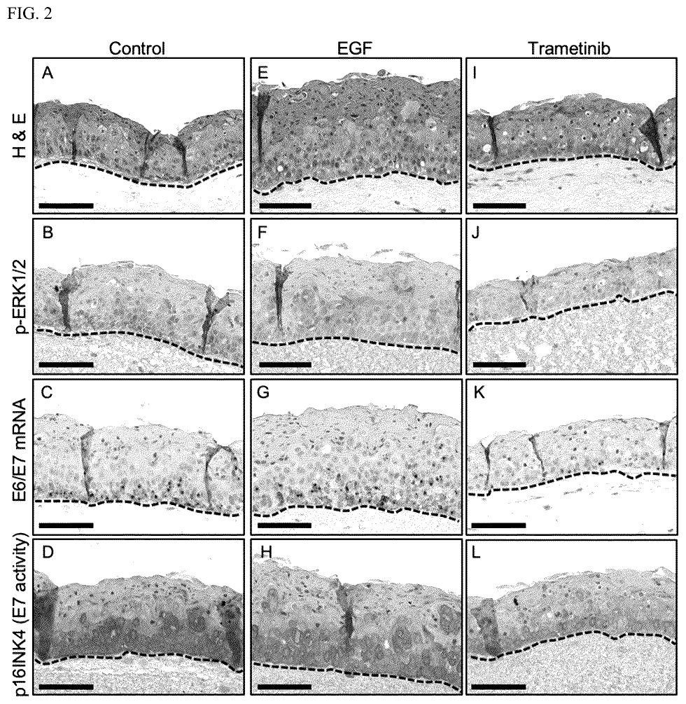

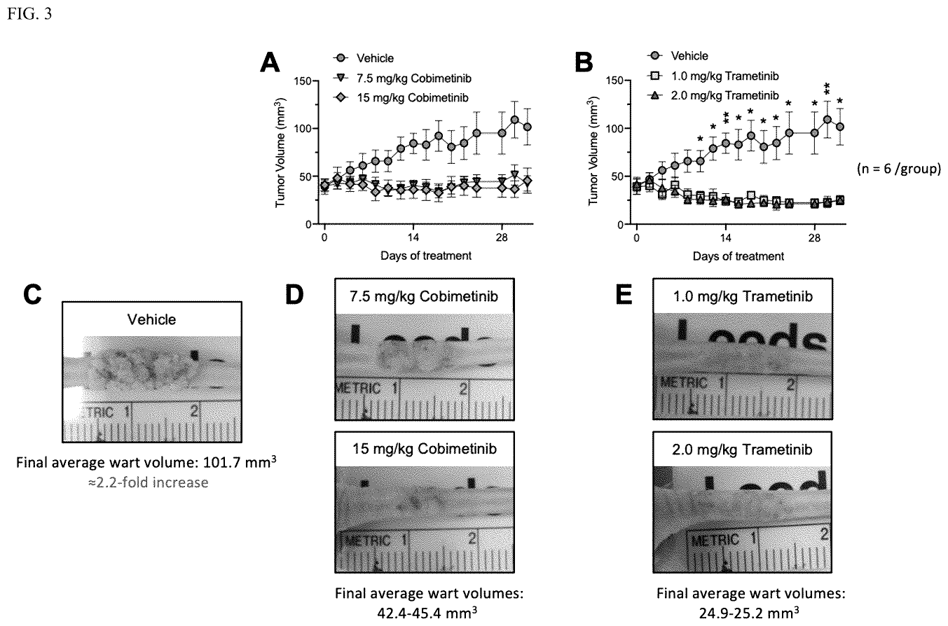

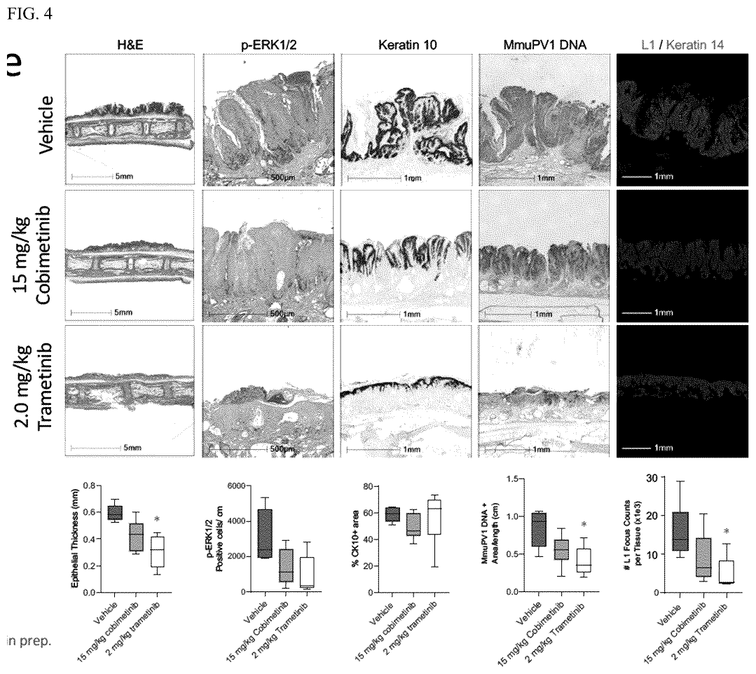

The mitogen-activated protein kinase kinase/extracellular signal-regulated kinase (MEK/ERK) protein kinase signaling cascade regulates HPV gene expression. HPV infection typically begins with an initial infected site in the basal epithelium and expands upwards through differentiating layers of the skin. Infection progression may be measured by increased MEK activation as measured by presence of phosphorylated ERK1/2 (p-ERK1/2) in progressively higher layers of the epithelium (FIG. 1). MEK inhibitors suppress production of HPV proliferation-inducing proteins E6 and E7, reducing hyperproliferation of infected cells. Small molecule inhibitors of various members of the MEK/ERK signaling cascade have been developed for the treatment of cancer. These MEK inhibitors could potentially be repurposed for treatment of HPV infection prior to and during cancer progression. Trametinib, an MEK inhibitor, suppresses hyperproliferation, production of E6/E7 mRNA, MEK activation, and E7 activity (as measured by the E7 surrogate, cellular p16INK4A protein) in a 3D tissue culture model of HPV oncogene transcription as shown in FIG. 2. As shown in FIG. 3, orally dosed trametinib suppresses wart grown in mice infected with a murine papilloma virus (MmuPV1). Mice infected with MmuPV1 receiving vehicle-only treatment developed a thick layer of tail warts (quantified in FIG. 3A, image in FIG. 3B), whereas mice infected with MmuPV1 receiving an oral dose of either 1 mg/kg or 2 mg/kg trametinib demonstrated regression of warts to a smaller size than those originally treated (FIG. 3A and FIG. 3C). Histology of treated animals supports these findings (FIG. 4).

Another MEK inhibitor, cobimetinib, has been shown to have similar effects. Cobimetinib differs from trametinib in that cobimetinib primarily inhibits MEK1, while trametinib effectively inhibits both MEK1 and MEK2. Cobimetinib suppresses wart growth but did not lead to regression (FIG. 3D). Histology of treated animals supports these findings (FIG. 4). Both cobimetinib and trametinib are of particular interest in treatment of papilloma viruses, but many other protein kinase inhibitors may be suitable as well.

Thus, while the description that follows occasionally describes compositions and methods in the context of exemplary embodiments in which the target of inhibition is MEK, the compositions and methods described herein can involve targeting any suitable target (e.g., a protein kinase or a transcription activator) involved in a cascade that regulates early viral (e.g., HPV) gene expression or viral genome replication. Exemplary alternative targets of inhibition include, but are not limited to, extracellular signal-regulated kinase (ERK), c-Jun N-terminal kinase (INK), protein kinase b (AKT), p38 MAPK, and activator protein-1 (AP-1).

Orally dosed trametinib has been approved by the FDA for treatment of melanoma, lung cancer, and thyroid cancer in combination with a B-Raf inhibitor (dabrafenib). However, like many other protein kinase inhibitors, oral trametinib is associated with substantial side effects, discouraging its use for non-malignant diseases. As such, alternate manners of administering MEK inhibitors that exploit the therapeutic effects of the inhibitor while avoiding the substantial side effects are desirable. For epithelial disorders, topical delivery of a therapeutic dose of one or more protein kinase inhibitors is one way to treat diseased tissue while limiting, or even eliminating, the effects of systemic delivery. Topical formulations suitable for precise application may expand use of protein kinase inhibitors (e.g., trametinib or other MEK inhibitors) for treating non-malignant epithelial disorders, precancerous lesions, or minimally invasive cancers.

Preparation of topical formulations for delivery of active ingredients for the treatment of papilloma viruses presents non-trivial complications. Papilloma virus replicative cycles are linked to differentiation of squamous epithelial cells, which are present in the basal (lowest) layer of the epithelium (FIG. 1). Topical delivery of protein kinase inhibitors for treating papilloma viruses can be challenging, as the formulation must penetrate the superficial cornified barrier of the skin to reach proliferating squamous epithelial cells. Traditional topical pharmaceutical compositions may not effectively deliver active ingredients to the basal layer of the epithelium. This disclosure provides topical formulations and methods for delivery of protein kinase inhibitors (e.g., MEK inhibitors) to differentiating squamous epithelial cells. The topical formulation and methods also may be applicable for delivery of transcription activators (e.g., AP-1). These formulations and methods may be suitable for treating proliferative or inflammatory skin diseases or disorders or skin diseases caused by infectious agents such as conditions described in more detail below.

Gene expression is frequently dysregulated in cells infected with HPV. Histone deacetylases (HDACs) are a class of enzyme that remove acetyl groups from histones. Histone acetylation is a known regulator of gene expression. Inhibition of histone deacetylases may regulate HPV gene expression and treat infection.

Examples of classes of HDAC inhibitors that may be of interest include hydroxamic acids, such as trichostatin A, cyclic tetrapeptides, such as trapoxin B, benzamides, electrophilic ketones, or aliphatic acids. Examples of specific HDAC inhibitors that may be of interest include vorinostat, belinostat, Panobinostat, entinostat, tacedinaline, mocetinostat, and nicotinamide. Topical formulation of these and other HDAC inhibitors may be of interest.

Another regulator of gene expression that may be of interest is transcriptional repressor CTCF. CTCF regulates gene expression by remodeling the structure of chromatin, binding a consensus sequence in genomic DNA. CTCF plays a role in many different cellular processes. Inhibition of CTCF may inhibit cell growth, inhibiting viral gene replication and halting or reversing progression of HPV infection.

CTCF inhibitors that may be of interest include azodicarbonamide, 3-nitrosobenzamide, 6-nitroso-1, 2-benzopyrone, 2,2′-di-thiobisbenzamide, bercaptobenzamides, N-[2-(5-pyridiniovaleroylthio) benzoyl]sulfacetamide bromide, and bis-thiadizolvenzene-1,2-diamine.

In one aspect, the present disclosure describes a pharmaceutical composition for topical delivery of one or more active pharmaceutical agents. In one or more embodiments, the pharmaceutical composition includes an effective amount of a protein kinase inhibitor or an inhibitor of a transcription activator implicated in viral replication (collectively, “active agent”), a gelation agent, and an aqueous solvent. A gelation agent is a polymer that forms a gel upon contact with a solvent. In one or more embodiments, the solvent may be added during preparation of the pharmaceutical composition. In one or more embodiments, the solvent may be a solvent boundary of a body. Examples of suitable solvent boundaries include an aqueous boundary or a mucous membrane.

In one or more embodiments, the gelation agent is a thermal gelation agent. A thermal gelation agent may only form a gel within a specific range of temperatures. In one or more embodiments, the thermal gelation agent forms a gel at a temperature of about 34° C. to about 40° C. Formation of a gel on a desired treatment site may improve delivery of a pharmaceutical agent, as the formulation may adhere to the site for a longer period of time than another pharmaceutical formulation, such as a lotion or cream. In one or more embodiments, a composition that gels at body temperature may be an isotonic composition.

In one or more embodiments, the pharmaceutical composition is a gel or forms a gel upon contact with an aqueous boundary. Examples of aqueous boundaries include, but are not limited to, a mucous membrane, the throat, the tonsils, the larynx, the respiratory tract, the mouth, the gums, the tongue, the eyes, the intestines, the anal/rectal site, the genitals, or any part of the skin. In one or more embodiments, when the pharmaceutical composition contacts a mucous membrane, it forms a mucoadhesive hydrogel. As used herein, the term “mucoadhesive,” “mucoadhesion,” and derivatives thereof refer to the attractive force between a mucous membrane and a material, such as the pharmaceutical composition of this disclosure. The mucous layer usually must be penetrated for an active ingredient to be delivered to cells. Forming a mucoadhesive hydrogel may improve delivery of the active agent to a treatment site. Improved delivery of an active agent may include improved penetration of the mucous membrane, delivery to deeper epithelial layers, a higher portion of the active agent being taken up by cells, or a combination thereof.

In one or more embodiments, the pharmaceutical composition forms a gel before it is applied to a treatment site. In some of these embodiments, the viscosity of the pharmaceutical composition may not change after application.

In one or more embodiments, the gelation agent may be a polymer. In one or more embodiments, the gelation agent may include gelatin, collagen, one or more polysaccharide (e.g., pectin, starch, tragacanth, etc.), one or more cellulose derivative (e.g., carboxymethyl cellulose (CMC), methyl cellulose (MC), hydroxyethyl cellulose (HEC), hydroxypropyl methylcellulose (HPMC), hydroxypropyl cellulose (HPC), ethyl hydroxyethyl cellulose (EHEC), and/or methyl hydroxyethyl cellulose (MHEC)), one or more polyacrylamide-based gelling agent (e.g., one or more carbomer), or one or more poloxamer.

In one or more embodiments, the gelation agent may include a thermal gelation agent such as, for example, a cellulose polymer. In one or more embodiments, the thermal gelation agent may be methylcellulose or a derivative thereof. In one or more embodiments, the thermal gelation agent may be hydroxypropyl methylcellulose or a derivative thereof. In one or more embodiments, the thermal gelation agent may be hydroxypropyl cellulose or a derivative thereof. The thermal gelation agent may include any suitable chemical substitutions. In one or more embodiments, the thermal gelation agent includes one or more propoxy groups, one or more methoxy groups, or a combination of one or more propoxy groups and one or more methoxy groups.

The gelation agent may have any suitable molecular weight. In one or more embodiments, the molecular weight of the gelation agent affects the viscosity of the gel formed. In one or more embodiments, forming a high viscosity gel may be desirable. In one or more alternative other embodiments, forming a low viscosity gel may be desirable. The viscosity of a gel may affect the rate at which the active agent is released. In one or more embodiments, the gelation agent may have a minimum molecular weight of at least 5 kilodaltons (kDa), at least 10 kDa, at least 20 kDa, at least 100 kDa, at least 200 kDa, at least 500 kDa, at least 850 kDa, at least 1,000 kDa, at least 10,000 kDa, at least 50,000 kDa, at least 100,000 kDa, at least 250,000 kDa, at least 500,000 kDa or at least 1,000,000 kDa. In one or more embodiments, the gelation agent may have a maximum molecular weight of no more than 200,000 kDa, no more than 100,000 kDa, no more than 50,000 kDa, no more than 15,000 kDa, no more than 10,000 kDa, no more than 5,000 kDa, or no more than 1,000 kDa. The gelation agent may have a molecular weight that falls within a range having endpoints defined by any minimum molecular weight described above and any maximum molecular weight that is greater than the selected minimum molecular weight. Thus, for example, the gelation agent may have a molecular weight of from 100 kDa to 200,000,000 kDa, from 5,000 kDa to 100,000 kDa, from 20 kDa to 15,000 kDa, etc.

In one or more embodiments, the pharmaceutical composition may include multiple gelation agents, each with a molecular weight independent of the molecular weight of any other gelation agent. For example, a pharmaceutical composition may include a portion of a gelation agent with a molecular weight of 100 kDa and a portion of a gelation agent with a molecular weight of 1,000 kDa. In one or more embodiments, the pharmaceutical composition may include at least one gelation agent and at least one thermal gelation agent.

The pharmaceutical composition may have any suitable viscosity. In one or more embodiments, the pharmaceutical composition may have a minimum viscosity of at least 1 centipoise (cps), at least 2 cps, at least 3 cps, at least 4 cps, at least 5 cps, at least 10 cps, at least 100 cps, at least 500 cps, at least 1,000 cps, at least 3,000 cps, at least 5,000 cps, at least 10,000 cps, or at least 100,000 cps. In one or more embodiments, the pharmaceutical composition may have a maximum viscosity of no more than 10,000,000 cps, no more than 1,000,000 cps, no more than 100,000 cps, no more than 50,000 cps, or no more than 5,000 cps. The pharmaceutical composition may have a viscosity that falls within a range having endpoints defined by any minimum viscosity listed above and any maximum viscosity listed above that is greater than the selected minimum viscosity. Thus, for example, the pharmaceutical composition may have viscosity of from 1 cps to 10,000,000 cps, from 3,000 cps to 100,000 cps, etc.

The viscosity of the pharmaceutical composition may change after it has been applied to an aqueous boundary. In one or more embodiments, the viscosity of the pharmaceutical composition may increase after it has been applied to an aqueous boundary. In one or more embodiments, the viscosity may increase a minimum of at least 1%, at least 5%, at least 10%, at least 20%, at least 30%, at least 50%, at least 100%, at least 500% or at least 1000% after it has been applied to an aqueous boundary. In one or more embodiments, the viscosity may increase by a maximum of no more than 10,000%, no more than 1000%, no more than 500%, no more than 100%, or no more than 50% after it has been applied to an aqueous boundary. In one or more embodiments, the viscosity may increase by an amount that falls with a range having endpoints defined by any minimum increase listed above and any maximum increased listed above that is greater than the selected minimum increase.

In one or more embodiments, the viscosity may increase a minimum of at least one-fold, at least two-fold, at least three-fold, at least five-fold, at least 10-fold, at least 50-fold, or at least 100-fold. In one or more embodiments, the viscosity may increase a maximum of no more than 1,000-fold, no more than 50-fold, no more than 10-fold, or no more than five-fold. In one or more embodiments, the viscosity may increase by an amount that falls with a range having endpoints defined by any minimum increase listed above and any maximum increase listed above that is greater than the selected minimum increase.

An effective amount of inhibitor in a topical gelling formulation may be different from a pharmaceutically active amount of the same inhibitor delivered orally or otherwise applied topically. In one or more embodiments, the pharmaceutical composition includes a minimum of at least 1 micromolar (μM), at least 2 μM, at least 5 μM, at least 10 μM, at least 20 μM, at least 30 μM, at least 50 μM, at least 100 μM, at least 200 μM, at least 300 μM, at least 400 μM, at least 500 μM, at least 600 μM, at least 700 μM, at least 800 μM, at least 900 μM, or at least 1 millimolar (mM) of the inhibitor. In one or more embodiments, the pharmaceutical composition includes a maximum of no more than 10 mM, no more than 5 mM, no more than 2 mM, no more than 1 mM, no more than 500 μM, or no more than 100 μM of the inhibitor. In one or more embodiments, the pharmaceutical composition includes an amount of inhibitor that falls with a range having endpoints defined by any minimum concentration listed above and any maximum concentration listed above that is greater than the selected minimum concentration. Thus, a pharmaceutical composition can include inhibitor at a concentration of, for example, from 1 μM to 1 mM, from 20 μM to 600 μM, from 5 μM to 500 μM, etc.

In one or more embodiments, the pharmaceutical composition includes a minimum of at least 0.001% by weight, at least 0.01% by weight, at least 0.02% by weight, at least 0.03% by weight, at least 0.04% by weight, at least 0.05% by weight at least 0.1% by weight at least 0.5% by weight, at least 1% by weight, at least 3% by weight, at least 5% by weight, at least 6% by weight, at least 10% by weight, or at least 15% by weight of the inhibitor. In one or more embodiments, the pharmaceutical composition includes a maximum of no more than 30% by weight, no more than 20% by weight, no more than 10% by weight, or no more than 5% by weight of the inhibitor.

The pharmaceutical composition of the present disclosure may be prepared in accordance with methods well known to the person skilled in the art of pharmaceutical formulation. The amount of the individual ingredients in the composition will, to some extent, depend on the concentration of the active ingredient incorporated therein. The amount of active ingredient in the composition may vary widely according to the severity of the condition to be treated, the age and condition of the patient and the discretion of the physician.

A kinase inhibitor may be selected to inhibit one or more kinases of interest. In one or more embodiments, the kinase inhibitor is an MEK inhibitor, an ERK inhibitor, a JNK inhibitor, an Aurora kinase inhibitor, or an inhibitor of another kinase implicated in viral replication.

An inhibitor of a transcription activator may be selected to inhibit one or more transcription activators of interest. In one or more embodiments, the inhibitor of a transcription activator inhibits AP-1 or another transcription activator implicated in viral replication.

In one or more embodiments, the MEK inhibitor is trametinib (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1 (2H)-yl}phenyl)acetamide), pyrrole derivatives, TAK-733 (one of a series of 8-methylpyrido[2,3-d]pyrimidine-4,7(3H,8H)-dione derivatives), CH4987655 and RDEA119/BAY 869766, cobimetinib ((S)-[3,4-Difluoro-2-(2-fluoro-4-iodophenylamino)phenyl][3-hydroxy-3-(piperidin-2-yl)azetidine-1-yl]methanone), binimetinib (5-((4-bromo-2-fluorophenyl)amino)-4-fluoro-N-(2-hydroxyethoxy)-1-methyl-1H-benzo[d]imidazole-6-carboxamide), selumetinib (6-(4-bromo-2-chloroanilino)-7-fluoro-N-(2-hydroxyelhoxy)-3-methylbenzimidazole-5-carboxamide), PD-325901, CI-1040, PD035901, tetrathiomolybtate, cobimetinib ((S)-[3,4-Difluoro-2-(2-fluoro-4-iodophenylamino)phenyl][3-hydroxy-3-(piperidin-2-yl)azetidine-1-yl]methanone), or TAK-933.

In one or more embodiments, the MEK inhibitor is trametinib, cobimetinib, or a combination thereof. In one or more embodiments, the composition may include more than one kinase inhibitor.

In one or more embodiments, the ERK inhibitor is a p-ERK1/2 inhibitor. In some of these embodiments, the p-ERK1/2 inhibitor is MK-8353 or CAS No. 1049738-54-6.

In one or more embodiments, the composition may include a pharmaceutically active ingredient in addition to the inhibitor. Examples of potential active ingredients include, but are not limited to, corticosteroids such as hydrocortisone; non-steroidal anti-inflammatories such as salicylic acid, salicylates, Vitamin D analogues (e.g., calcipotriol), immunophilins, p38 kinase inhibitors, and/or calcineurin inhibitors (e.g., tacrolimus and pimecrolimus); cannabinoids; vasomodulators (e.g., alpha adrenoreceptor ligands); topical anesthetics including but not limited to bupivacaine, chlorprocaine, dibucaine, ketamine, and/or pramoxine; anti-infectives including but not limited to topical antibiotics (e.g., clindamycin); antifungals; antivirals; histamine H1 receptor antagonists; histamine H2 receptor antagonists; histamine H3 receptor antagonists; leukotriene antagonists including but not limited to antagonists of LTB4, LTC4, LTD4, and/or LTE4 (e.g., montelukast); phosphodiesterase inhibitors including but not limited to PDE3 inhibitors, PDE4 inhibitors, PDES inhibitors, PDE7 inhibitors and/or inhibitors of two or more phosphodiesterases, (e.g., dual PDE3/PDE4 inhibitors); neurotransmitter re-uptake inhibitors including but not limited to fluoxetine, sertraline, paroxetine, and/or ziprasidone; 5-lipoxygenase (5-LO) inhibitors or 5-lipoxygenase activating protein (FLAP) inhibitors; α1- and α2-adrenoceptor agonist vasoconstrictor sympathomimetic agents; muscarinic M3 receptor antagonists or anticholinergic agents; 02-adrenoceptor agonists; dual acting 02/M3 agents; xanthines (e.g., theophylline and/or aminophylline); non-steroidal anti-inflammatories including but not limited to sodium cromoglycate and/or nedocromil sodium; ketotifen; COX-1 inhibitors (NSAIDs) and COX-2 selective inhibitors; oral, inhaled, intranasal or topical glucocorticosteroids; monoclonal antibodies active against endogenous inflammatory entities; anti-tumor necrosis factor (anti-TNF-α) agents; immunosuppressive agents; inhibitors of matrix metalloproteases (MMPs); tachykinin NK1, NK2 and NK3 receptor antagonists; elastase inhibitors; adenosine A2a receptor agonists; inhibitors of urokinase; compounds that act on dopamine receptors (e.g., D2 agonists); modulators of the NF-κB pathway (e.g., IKK inhibitors); agents that can be classed as mucolytics or anti-tussive agents; antibiotics; modulators of cytokine signaling pathways including but not limited to p38 MAP kinase inhibitors, SYK kinase inhibitors or JAK kinase inhibitors; modulators of the prostaglandin pathways including but not limited to inhibitors of H-PDGS and antagonists of DP-1 and CRTH2; antagonists of chemokine receptors CXCR1 and CXCR2; antagonists of chemokine receptors CCR3, CCR4 and/or CCR5; inhibitors of phosphoinositide-3-kinase; HDAC inhibitors; p38 inhibitors; CXCR2 antagonists; calcineurin inhibitors; anti-interleukin 17 (anti-IL-17) agents; anti-interleukin 4 receptor (anti-IL4R) agents; or anti-interleukin 31 (anti-IL-31) agents; PD-L1 inhibitors; other agonists of T-cells; agonists of TLK47; interferons; activators of interferon-stimulated genes; miglustat; eliglustat; inhibitors of RNA binding protein HuR; paeoniflorin and/or paeoniflorin-6′-O-benzene sulfonate CP25; phosphor-BRD4 targeting compounds; DC1-1; DC-2; activators of the DREAM complex; diazoxide; inhibitors of ATP-sensitive potassium channels; hippo; YAP/TAX inhibitors; ivermectin; YAP/TAZ inhibitor-1; TEAD inhibitors such as MYF-03-69; ubiquitination inhibitors; inhibitors of E3 ubiquitin-protein ligase MARCHF8; and/or inhibitors of SIRT1 (e.g., EX-27).

Pharmaceutically acceptable salts of the MEK inhibitors having an acidic moiety can be formed from organic and inorganic bases. Suitable salts with bases are, for example, metal salts, such as alkali metal or alkaline earth metal salts, for example sodium, potassium, or magnesium salts; or salts with ammonia or an organic amine, such as morpholine, thiomorpholine, piperidine, pyrrolidine, a mono-, di- or tri lower alkylamine, for example ethyl-tert-butyl-, diethyl-, diisopropyl-, triethyl-, tributyl- or 5 dimethylpropylamine, or a mono-, di-, or trihydroxy lower alkylamine, for example mono-, di- or triethanolamine.

In one or more embodiments, the pharmaceutical composition includes a minimum of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 90% of an aqueous solvent by weight. In one or more embodiments, the pharmaceutical composition includes a maximum of no more than 100%, no more than 98%, no more than 95%, no more than 92%, no more than 90%, no more than 88%, no more than 85%, no more than 82%, no more than 80%, no more than 70%, or no more than 60% of an aqueous solvent by weight. The aqueous solvent may include water, alcohol, or a combination thereof. In one or more embodiments, the alcohol may be an ethoxy alcohol, such as 2-(2-ethoxyethoxy)ethanol (TRANSCUTOL, Gattefosse SAS, Saint-Priest, France).

While some protein kinase inhibitors are most soluble in organic solvents, such as DMSO, inclusion of organic solvents in a pharmaceutical composition for topical application may be undesirable. For example, while a solvent such as DMSO may improve solubility of an MEK inhibitor such as trametinib, it may irritate skin. Organic solvents such as DMSO may additionally enable penetration of protein kinase inhibitors such as trametinib through the epithelium to the dermal layers. For one or more applications described in this disclosure, delivery to the dermal layers of the skin may be undesirable. Additionally, while many organic solvents may be used in animal models of disease, many are restricted from human use. Therefore, any pharmaceutical composition demonstrated to work in an animal model that includes an organic solvent may require reformulation for human use regardless of efficacy.

Thus, aqueous formulation of protein kinase inhibitors presents suitable challenges, as many protein kinase inhibitors, including MEK inhibitors, are not soluble in aqueous solvents. To improve solubility of an inhibitor (protein kinase inhibitor or otherwise) in a pharmaceutical composition, a solubilizer may be included. Any non-toxic solubilizer may be suitable for inclusion in the pharmaceutical composition. In one or more embodiments, the solubilizer may be an alcohol, ether alcohol, esters, fatty acid, non-ionic surfactant, anionic surfactant, and or cationic surfactant. In one or more embodiments, the solubilizer may be selected from ethanol, isopropyl alcohol, octyl dodecanol, oleyl alcohol, diethylene glycol monoethyl ether (DGME), dipropylene glycol, propylene glycol, 1,2-butylene glycol, ethyl oleate, glyceryl monooleate, glyceryl monocaprate, isopropyl palmitate, isopropyl myristate, propylene glycol monolaurate, propylene glycol monocaprylate, caprylate, caprate, laurate, linolate, oleate, palmitate, stearate, isostearate, BRIJ (Croda Americas LLC, Wilmington, DE), LABRASOL (Gattefosse SAS, Saint-Priest, France), LABRAFIL (Gattefosse SAS, Saint-Priest, France), TWEEN 80, TRANSCUTOL (Gattefosse SAS, Saint-Priest, France), azone, oleic acid, N-methyl-pyrrolidone, 1-2-pentanediol, 1-5 pentanediol, 1-3 butanediol, 1-3 glycerol, dimethyl isosorbide, sodium lauryl sulfate, an alkyl ammonium halide, an amide, a terpene, a sulfoxide, or any combination of two or more of the foregoing.

In one or more embodiments, the pharmaceutical composition can include a cosolvent. Exemplary cosolvents include, but are not limited to, glycerin (glycerol), polyethylene glycol (PEG), benzyl alcohol, triacetin (glyceryl triacetate), ethyl lactate, castor oil, coconut oil, a caprylic/capric triglyceride, urea, menthol, lecithin, polyoxyl-35 castor oil (castor oil polyoxyethylene ether), linoleic acid, eucalyptol, squalene, or a combination of any two or more of the foregoing.

The pharmaceutical compositions of this disclosure use alternate approaches for solubilizing the protein kinase inhibitor that typically do not involve organic solvents. In one or more embodiments, the pharmaceutical composition does not include an organic solvent. When an organic solvent is present, the pharmaceutical composition includes no more than 10%, no more than 9%, no more than 8%, no more than 7%, no more than 6%, no more than 5%, no more than 4%, no more than 3%, no more than 2%, no more than 1%, no more than 0.5%, no more than 0.1%, or no more than 0.01% organic solvent by weight. Organic solvents may include, but are not limited to, hexanol, acetone, toluene, n-hexane, heptane, dichloromethane, methanol, benzene, xylene, ethyl acetate, chloroform, diethyl ether, tetrahydrofuran, acetonitrile, dimethylformamide, dimethyl sulfoxide and formaldehyde.

In one or more embodiments, the pharmaceutical composition does not include an irritating compound in an amount effective to cause irritation. That is, an irritating compound may be present in the composition in trace amounts or otherwise present in an amount less than necessary to exhibit irritating effects. Irritation caused by a carrier or solvent is a factor to be considered when preparing compositions for topical administration. It may be desirable for the pharmaceutical composition to omit any compound that may irritate the skin. Examples of irritating compounds include, but are not limited to, an aldehyde, a strongly alkaline solution, a strongly acidic solution, a detergent, or an adhesive.

Inclusion of an adhesion promoter may improve delivery of the inhibitor and/or penetration through multiple layers of the epidermis. In one or more embodiments, the pharmaceutical composition of includes an adhesion promoter.

The pharmaceutical composition may include a drug release inhibitor that regulates release of the active agent, thereby controlling the duration over which the active ingredient is released from the pharmaceutical composition.

It may be desirable to include additional components of the pharmaceutical composition to modify physical properties of the composition or the gel or film formed thereof. In one or more embodiments, the pharmaceutical composition may include an anti-tacking agent. In one or more embodiments, the pharmaceutical composition may include a gel strength modifier. In one or more embodiments, the pharmaceutical composition may include a gelling aid. The gel may additionally include any other excipients as desirable, such as colorants, preservatives, and fillers.

Exemplary anti-taking agents include, but are not limited to, talc, magnesium stearate, silicon dioxide, stearic acid, calcium stearate, glyceryl stearate, microcrystalline cellulose, carnauba wax, polyethylene glycol (PEG), sodium lauryl sulfate, calcium silicate, titanium dioxide, hydrogenated vegetable oil, zinc stearate, polyvinyl alcohol (PVA), starch (e.g., pre-gelatinized or native), isopropyl myristate, methylcellulose, polyacrylate polymers, kaolin, corn starch, sucrose ester, cellulose derivatives (e.g., hydroxypropyl methylcellulose), gelatin, dimethicone, shellac, ethylcellulose, propylene glycol, hydroxypropylcellulose (HPC), acacia gum, or any combination of two or more of the foregoing. Anti-tacking agents help reduce stickiness of the product that may otherwise affect manufacturing or application of the product.

Gel strength modifiers adjust the mechanical properties, viscosity, and firmness of gels. Gel strength modifiers can be used to either increase or decrease the gel strength, depending on the desired strength of the gel. Gel strength modifiers are selected based on the type of gel (e.g., aqueous or non-aqueous), compatibility with components of the gel (e.g., active agents), and/or the desired texture, spreadability, and/or stability of the formulation. Exemplary gel strength modifiers include, but are not limited to, a carbomer, xanthan gum, hydroxypropyl methylcellulose (HPMC), hydroxyethylcellulose (HEC), methylcellulose, a poloxamer, gelatin, pectin, guar gum, sodium alginate, agar, carrageenan, an acrylate/C10-30 alkyl acrylate crosspolymer, hyaluronic acid, polyvinyl alcohol (PVA), chitosan, tragacanth, starch, polyethylene glycol (PEG), collagen, magnesium aluminum silicate, locust bean gum, konjac gum, tamarind seed polysaccharide, a cellulose derivatives (e.g., CMC, HPC), or any combination of two or more of the foregoing. In general, gel strength modifiers may be used to help regulate release of active agent from the gel. In general, a gel with greater strength will release an active agent more slowly than a gel with less strength. In one or more embodiments, the gel may have a strength of 11-65 Pascals.

Gelling aids are substances that enhance or facilitate the gelling action of primary gelling agents, often by adjusting the pH, ion concentration, or enhancing the interaction between polymer molecules. For example, one can enhance interaction between polymer molecules by dehydrating certain groups in a cellulose ether polymer gel. Exemplary gelling aids include, but are not limited to, triethanolamine (TEA), sodium hydroxide, potassium hydroxide, ethyl alcohol, propylene glycol, glycerin (glycerol), sorbitol, tromethamine (Tris), boric acid, sodium bicarbonate, ammonium hydroxide, phosphoric acid, acetic acid, lactic acid, citric acid, tetrasodium EDTA, an ionic salt (e.g., sodium chloride (NaCl), sodium fluoride (NaF), calcium chloride (CaCl₂)), potassium chloride (KCl), magnesium chloride (MgCl₂), sodium sulfate (Na₂SO₄), potassium sulfate (K₂SO₄), calcium sulfate (CaSO₄), magnesium sulfate (MgSO₄), sodium phosphate (Na₃PO₄), potassium phosphate (K₃PO₄), sodium citrate (Na₃C₆H₅O₇), ammonium sulfate ((NH₄)₂SO₄, etc.), or any combination of two or more of the foregoing.

In another aspect, this disclosure describes ways in which the pharmaceutical composition described herein may be delivered. In one or more embodiments, it may be desirable to provide the pharmaceutical composition in a dehydrated or desiccated form to be mixed with a solvent directly before use. In one or more embodiments, the pharmaceutical composition may be provided as a prepared gel. In one or more embodiments, the pharmaceutical composition may be provided in a form intended to form a gel upon application to at a treatment site. In order to form a gel on a treatment site, it may be desirable to deliver or apply a thin film of the composition.

The various embodiments of active agent delivery devices described herein can be utilized in any suitable application, e.g., transdermal or topical active agent delivery and medical wound dressings, etc. In one or more embodiments where the treatment site is on the skin, the pharmaceutical composition may be provided in a container with an applicator. The applicator may be a brush, a dropper, a sponge, perforated cap, a pump, or any other suitable applicator. The container may be suitable for direct application of the formulation to the site of interest, such as a stick or a compressible tube. In one or more embodiments, the pharmaceutical composition may be provided in the form of a bandage loaded with the pharmaceutical composition. In one or more embodiments, the pharmaceutical composition may be provided in the form of an ointment to be applied. The ointment may be provided with (e.g., in contact with, incorporated into, etc.), for example, a bandage, a wrap, a plaster, a dressing, etc. In one or more embodiments, the pharmaceutical composition may be provided as a spray. It may be desirable to apply the pharmaceutical composition to an air pathway, such as the lungs or larynx. In one or more embodiments, the pharmaceutical composition may be provided in an inhaler, such as a metered dose inhaler. In one or more embodiments, the pharmaceutical composition may be provided in the form of a tablet, such as a gel tablet or a dissolvable tablet. The tablet may be administered orally or as a suppository.

### Methods of Treatment

In another aspect, this disclosure describes a method of treating an epithelial disorder in a subject in need thereof, the method including applying a pharmaceutically active amount of any of the pharmaceutical compositions described herein to a treatment site. In one or more embodiments, the treatment site is on a mucous membrane or an epidermal surface. In one or more embodiments, the treatment site is on the skin, oral cavity, pharyngeal cavity, larynx, airway, vagina, cervix, genitals, or anus/rectum.

In one or more embodiments, the pharmaceutical composition may be dried for storage, then reconstituted by rehydrating the composition prior to administering the composition to a subject. In other embodiments, a dried composition may be administered to a treatment site and be reconstituted or rehydrated in situ.

As described herein, there are multiple diseases that may benefit from epithelial delivery of an inhibitor of certain viral proliferative functions (e.g., protein kinases and/or transcription activators implicated in viral replication). While described herein in the context of an exemplary embodiment in which the pharmaceutical composition is used to treat infection by HPV, the compositions and methods described herein may involve treating many other diseases. In one or more embodiments, the compositions and methods described herein are suitable for treatment of epithelial diseases. In one or more embodiments, the compositions and methods described herein are suitable for treatment of viral diseases. In one or more embodiments, the compositions and methods described herein are suitable for treatment of autoimmune diseases. In one or more embodiments, the compositions and methods described herein are suitable for treatment of cancer. In one or more embodiments, the compositions and methods described herein may be suitable for treatment of human papilloma virus, eczema, psoriasis, atopic dermatitis, seborrheic dermatitis, histaminergic itch, acne, rosacea, hives, alopecia, shingles, chicken pox, cold sores, herpes infections (e.g., genital herpes, ocular herpes, herpes gladiatorum, etc.), Chlamydia infection, Gonorrhea infection, pelvic inflammatory disease, poxvirus infections (e.g., monkey pox), molluscum contagiosum, viral warts (e.g., HPV cutaneous warts, HPV genital warts, etc.), HPV neoplasia (e.g., in the oral cavity, genitals, genital tract, etc.), trichodysplasia spinulosa (polyomavirus), or non-viral warts (e.g., seborrheic keratosis).

In one or more embodiments, the epithelial disease is a skin cancer or a skin hyperplasia such as, for example, squamous cell carcinoma (SCC), adenocarcinoma (endocervix), basal cell carcinoma (BCC), hyperkeratosis, actinic keratosis, sebaceous adenoma, UV induced skin cancer, syringoma, Merkel cell carcinoma, keratinocytic epidermal nevi, nevi sebacei, etc. In one or more embodiments, the epithelial disease is a hyperplastic condition affecting nervous epithelial tissue such as, for example, neurofibromatosis type 1 (NF1). As used herein, “hyperplasia,” “hyperplastic condition,” and variants thereof refer generically to conditions characterized by an increase in cell number in an organ or tissue. Cells in a hyperplastic condition typically appear normal under microscopy. Thus, a hyperplastic condition is typically not considered cancerous, but may become cancerous if left untreated. Topical treatment of such conditions with an MEK inhibiting agent is desirable to reduce side effects of active ingredients.

In one or more embodiments wherein the skin disorder is a viral disease, the viral disease is part of a family selected from Poxviridae, Herpesviridae, Adenoviridae, Anelloviridae, Flaviviridae, Papillomaviradae, Polyomaviridae, Parvoviridae, Orthomyxoviridae, Paramyxoviridae, Picornaviriade, Matonaviridae, or Retroviridae.

In one or more embodiments wherein the skin disorder is a bacterial disease, the bacterial disease is part of a family selected from Chlamydiaceae, Neisseriaceae, Staphylococcaceae, Streptococcaceae.

In one or more embodiments, the skin disorder is selected from the group comprising scarring, dermatitis, a proliferative disease or condition, a mast cell disease or condition, a burn or contact with an allergen and/or an irritant.

In one or more embodiments, the skin disorder is selected from the group comprising atopic dermatitis, bullous disorders, collagenases, psoriasis, psoriatic lesions, seborrheic dermatitis or contact dermatitis, eczema, urticaria, pruritus, rosacea, prurigo nodularis, hypertrophic scarring, keloid scar formation, scleroderma, Folliculitis keloidalis nuchae, Kawasaki Disease, Sjogren-Larsson Syndrome, Grover's disease, a first degree burn, a second degree burn, a third degree burn, a fourth degree burn, cutaneous mucinosis, solar keratosis, squamous cell carcinoma or melanoma, asteatotic eczema, discoid eczema, hand eczema, gravitational/varicose eczema, eczematous drug eruptions, lichen simplex, lichen sclerosus, lichen planus Irritant, allergic contact dermatitis, photoallergic/photoaggravated dermatitis, infective (secondary to bacterial/viral/fungal infection) dermatitis, pruritic diseases including those associated with chronic systemic disorders such as uremic pruritus, cholestatic pruritus, adult blaschkitis, aquadynia, aquagenic pruritus, balsam of Peru, biliary pruritus, brachioradial pruritus, drug-induced pruritus, hydroxyethyl starch-induced pruritus, itchy points, lichen simplex chronicus, neurodermatitis, prion pruritus, prurigo, prurigo pigmentosa, prurigo simplex, pruritus ani, pruritus scroti, pruritus vulvae, puncta pruritica, referred itch, renal pruritus, scalp pruritus, senile pruritus, xerotic eczema, itch associated with HIV infection, T-cell lymphoma, Sezary syndrome, mycosis fungoides, or trichodysplasia spinulosa.

The subject treated by the methods described herein may be any subject in need of treatment. Most commonly, the subject is a mammal, such as a human, a mouse, a dog, a cat, a pig, a primate, a sheep, a bovine, a rabbit, a ferret, or a rat. The subject may have other diseases and/or comorbidities that affect their treatment strategy. Patients with HPV who additionally have a disease that requires immunosuppressants, such as HIV or organ transplantation, may be at higher risk for HPV-related complications, and therefore more in need of topical treatment (either therapeutic or prophylactic) of HPV-infected tissue. In one or more embodiments, the subject is immunosuppressed. In one or more embodiments, the subject does not have known mutation to a BRAF gene.

The use of a protein kinase inhibitor and/or an inhibitor of a transcription activator to treat viral infections may act independently from the immune system. For example, some protein kinase inhibitors directly affect cell division and inhibit viral replication within an individual cell and without immune intervention. Similarly, an inhibitor of a transcription factor implicated in viral replication inhibit viral replication within an individual cell and without immune intervention. In one or more embodiments, the methods described herein treat viral infection in the absence of an immune system. In one or more embodiments, the methods described herein treat viral infection in immune privileged tissues. Immune privileged tissues may include the brain, the epithelium, the uterus during pregnancy, testes, eyes, and spinal cord. In one or more embodiments, the methods disclosed herein are suitable for treatment of papilloma virus infection of immune privileged tissues.

The methods disclosed herein may be described by their effect on the pharmacokinetics of a delivered protein kinase inhibitor or delivered inhibitor of a transcription activator implicated in viral replication (collectively, “active agent”). Examples of pharmacokinetic properties that may be altered by delivering an active agent using a gelation agent include the required dose, dosing interval, Cₘₐₓ, tₘₐₓ, concentration of active agent in the bloodstream, absorption rate constant, area under the curve, and fluctuation. It may be desirable to prepare formulations for topical use that increase delivery of the active agent to the skin while decreasing the systemic concentration of the active agent in the bloodstream. Additionally, it may be desirable to slow absorption of the active agent from the gel into the skin, which may require fewer doses than an active agent administered topically as a cream to achieve a similar duration of effect. In this sense, administering an active agent in a formulation that includes a gelation agent may increase the dosing interval relative administering the active agent in a formulation without a gelation agent. Administering an active agent in a formulation that includes a gelation agent may require a lower dose to be administered to a subject over an extended period of time relative to administering the active agent in a formulation without a gelation agent.

As described herein, inclusion of a gelation agent may affect the rate of active agent liberation and absorption. Pharmaceutical compositions including an active agent and a gelation agent may exhibit a delayed or extended release of active agent into the skin as compared to topical compositions not including a gelation agent. In one or more embodiments, treatment using the methods described herein results in active agent release at least one, at least two, at least four, at least six, at least 12, at least 24, at least 48, at least 72, at least 96, or at least 120 hours after application of the pharmaceutical composition. Inclusion of a gelation agent may also affect the amount of active agent that permeates the deep endothelial cells, and the amount of active agent that is absorbed in the bloodstream. Pharmaceutical compositions including a gelation agent may exhibit lower levels of systemic delivery of active agent as measured by bloodstream levels of active agent relative to compositions that do not include a thermal gelation agent. In one or more embodiments, treatment using the methods described herein results in no more than 80%, no more than 70%, no more than 60%, no more than 50%, no more than 40%, no more than 30%, no more than 20%, or no more than 10% of the bloodstream level of active agent relative to topical treatment with a formulation that does not include a gelation agent. In one or more embodiments, treatment using the methods described herein results in at least 1%, at least 5%, at least 10%, or at least 20% of the bloodstream level of active agent relative to topical treatment with a formulation that does not include a gelation agent. In one or more embodiments, treatment using the methods described herein results in a maximum bloodstream level of active agent of no more than 100 g/mL, no more than 10 g/mL, no more than 100 ng/mL, no more than 10 ng/mL or no more than 1 ng/mL of actie agent in the bloodstream. In one or more embodiments, treatment using the methods described herein results in a minimum bloodstream level of active agent of at least 1 pg/mL, at least 10 pg/mL, or at least 100 pg/mL. Bloodstream levels of active agent may be measured by any suitable method, including ELISA, HPLC, or colorimetric assay.

The methods described herein may be characterized by their effect on a treated area. In one or more embodiments, the methods described herein result in decreased phosphorylation of ERK in the treated area relative to a comparable untreated area. Phosphorylation of ERK may be measured by any suitable method including immunoblotting, ELISA, or immunohistochemistry. In one or more embodiments, the methods described herein result in decreased levels of p-ERK in the treated area relative to a comparable untreated area. Abundance of p-ERK may be measured by any suitable method including Immunoblotting, ELISA, or immunohistochemistry.

In embodiment where the treated area is infected with a virus, the methods described herein may decrease the amount of viral transcription occurring in a treated area. In one or more embodiments, the methods described herein may decrease viral transcription in a treated area by a minimum of at least one-fold, at least two-fold, at least four-fold, at least six-fold, at least 10-fold, or at least 100-fold relative to a comparable untreated area. In one or more embodiments, the methods described herein may decrease viral transcription in a treated area by a maximum of no more than 200-fold, no more than 100-fold, no more than 50-fold, no more than 30-fold, or no more than 10-fold relative to a comparable untreated area.

In one or more embodiments, the subject treated by the methods described herein may have a bacterial infection. The bacterial infection may be symptomatic, or it may be asymptomatic. In one or more of these embodiments, treatment with the compositions described herein may result in decreased bacterial burden. The methods described herein may reduce bacterial burden by at least 10%, at least 25%, at least 50%, at least 60%, at least 70%, at least 80%, at least 90%, or 100%. Bacterial burden may be measured using any standard method, such as qPCR, immunoblot, or sample culture.

In one or more embodiments, the methods described herein may result in decreased tumor volume. Decreased tumor volume may be defined as either a reduction in the volume of a pre-existing tumor, or as development of a smaller tumor relative to an untreated subject or area. In one or more embodiments, the methods described herein decrease tumor volume by a minimum of at least 5%, at least 10%, at least 50%, or at least 100%.

In another aspect, this disclosure describes a kit for topical treatment of an epithelial disorder including the compositions described herein. In one or more embodiments, the kit includes a pharmaceutical composition consistent with those described herein and instructions for use.

A kit may include one or more containers filled with one or more of the compositions of this disclosure. Additionally, the kit may include other reagents such as buffers and solutions needed to utilize the compositions or practice the methods described herein are also included. Optionally associated with such container(s) may be a notice or printed instructions. As used herein, the phrase “packaging material” refers to one or more physical structures used to house the contents of the kit. The packaging material is constructed by well-known methods, preferably to provide a sterile, contaminant-free environment. As used herein, the term “package” refers to a solid matrix or material such as glass, plastic, paper, foil, and the like, capable of holding within fixed limits a pharmaceutical composition including a gelation agent and an active pharmaceutical ingredient.

The composition described herein may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the active agent without causing any undesirable biological effects or interacting in a deleterious manner with any of the other components of the pharmaceutical composition in which it is contained.

A single dose may be administered all at once, continuously for a prescribed period of time, or in multiple discrete administrations. When multiple administrations are used, the amount of each administration may be the same or different. For example, a dose of 1 mg per day may be administered as a single administration of 1 mg, continuously over 24 hours, as two or more equal administrations (e.g., two 0.5 mg administrations), or as two or more unequal administrations (e.g., a first administration of 0.75 mg followed by a second administration of 0.25 mg). When multiple administrations are used to deliver a single dose, the interval between administrations may be the same or different.

In one or more embodiments, the pharmaceutical composition may be administered, for example, from a single dose to multiple doses per week, although in one or more embodiments the method can involve a course of treatment that includes administering doses of the pharmaceutical composition at a frequency outside this range. When a course of treatment involves administering multiple doses within a certain period, the amount of each dose may be the same or different. For example, a course of treatment can include a loading dose initial dose, followed by a maintenance dose that is lower than the loading dose. Also, when multiple doses are used within a certain period, the interval between doses may be the same or be different.

In certain embodiments, the pharmaceutical compositions of this disclosure may be administered from about once per month to about five times per week.

A component is said to be present in amounts “no more than” a reference amount or concentration when the component is not absent but is present in an amount up to the reference amount or concentration.

“At risk” refers to a subject that may or may not actually possess the described risk. Thus, for example, a subject “at risk” of infection by a microbe is a subject present in an area where individuals have been identified as infected by the microbe and/or is likely to be exposed to the microbe even if the subject has not yet manifested any detectable indication of infection by the microbe and regardless of whether the subject may harbor a subclinical amount of the microbe.

A “cellular signaling pathway” refers to a cascade of biochemical activity that biochemically links an agonist-receptor interaction with a cellular response to the agonist-receptor binding (e.g., cytokine production).

As used herein, the term “active agent” is used to collectively refer to a protein kinase inhibitor and/or an inhibitor of a transcription activator implicated in viral replication. Thus, the term used in the singular includes the plural.

“Inhibit” and variations thereof refer to any measurable reduction of cellular activity. For example, inhibition of a particular protein kinase refers to a decrease in activity of the protein kinase. As another example, inhibition of a transcription activator refers to a decrease in transcription of a nucleotide sequence otherwise transcribed to a greater degree in the absence of the transcription activator being inhibited. The extent of inhibition may be characterized as a percentage of a normal level of activity.

“Treat” or variations thereof refer to reducing, limiting progression, ameliorating, or resolving, to any extent, the symptoms or signs related to a condition. A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after the condition manifests in a subject, while “prophylactic” treatment is initiated before a condition manifest in a subject.

A “effective amount” of a compound refers to an amount sufficient to induce a desired therapeutic or prophylactic effect. For a given compound, there may be a range of amounts considered “effective.” An amount sufficient to induce the desired therapeutic or prophylactic effect with minimal side-effects may be the most effective in some circumstances.

“Treatment site” refers to the site of a particular treatment. Depending upon the particular treatment, the treatment site may be an entire organism (e.g., a systemic treatment) or any portion of an organism (e.g., a localized treatment).

As used herein, the word “exemplary” means to serve as an illustrative example and should not be construed as preferred or advantageous over other embodiments.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

In the preceding description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

In several places throughout the above description, guidance is provided through lists of examples, which examples can be used in various combinations. In each instance, the recited list serves only as a representative group and should not be interpreted as an exclusive list.

For any method disclosed herein that includes discrete steps, the steps may be performed in any feasible order. And, as appropriate, any combination of two or more steps may be performed simultaneously.

### EXEMPLARY EMBODIMENTS

Embodiment 1 is pharmaceutical composition comprising:
a protein kinase inhibitor or an inhibitor of a transcription activator implicated in viral replication;
a gelation agent; and
an aqueous solvent.

Embodiment 2 is the pharmaceutical composition of Embodiment 1, wherein the gelation agent is a cellulose polymer.

Embodiment 3 is the pharmaceutical composition of Embodiments 1 or 2, wherein the gelation agent comprises hydroxypropyl methylcellulose, methylcellulose, or hydroxypropyl cellulose.

Embodiment 4 is the pharmaceutical composition of any of Embodiments 1 to 3, wherein the gelation agent has a molecular weight of 10 kDa to 2,000 kDa.

Embodiment 5 is the pharmaceutical composition of any of Embodiments 1 to 4, wherein the gelation agent has a viscosity range of 100 cps to 10,000 cps.

Embodiment 6 is the pharmaceutical composition of any of Embodiments 1 to 5, wherein the composition forms a gel upon contact with an aqueous boundary.

Embodiment 7 is the pharmaceutical composition of any of Embodiments 1 to 6, wherein the kinase inhibitor is an MEK inhibitor or an ERK inhibitor.

Embodiment 8 is the pharmaceutical composition of Embodiment 7, wherein the MEK inhibitor is binimetinib, cobimetinib, selumetinib, or trametinib.

Embodiment 9 is the pharmaceutical composition of Embodiment 8, wherein the MEK inhibitor is trametinib.

Embodiment 10 is the pharmaceutical composition of any of Embodiments 1-9, further comprising a solubilizer.

Embodiment 11 is the pharmaceutical composition of Embodiment 10, wherein the solubilizer comprises an alcohol, an ether alcohol, an ester, a fatty acid, a non-ionic surfactant, an anionic surfactant, or a cationic surfactant.

Embodiment 12 is the pharmaceutical composition of Embodiments 10 or 11, wherein the solubilizer is selected from ethanol, isopropyl alcohol, octyl dodecanol, oleyl alcohol, diethylene glycol monoethyl ether, dipropylene glycol, propylene glycol, 1,2-butylene glycol, ethyl oleate, glyceryl monooleate, glyceryl monocaprate, isopropyl palmitate, isopropyl myristate, propylene glycol monolaurate, propylene glycol monocaprylate, caprylate, caprate, laurate, linolate, oleate, palmitate, stearate, isostearate, BRIJ, LABRASOL, LABRAFIL, TWEEN 80, TRANSCUTOL, sodium lauryl sulfate, alkyl ammonium halides, amides, terpenes, and sulfoxides.

Embodiment 13 is the pharmaceutical composition of any of Embodiments 1 to 12, wherein the aqueous solvent is water.

Embodiment 14 is the pharmaceutical composition of any of Embodiments 1 to 13, wherein the aqueous solvent is at least 40% of the pharmaceutical composition by weight.

Embodiment 15 is the pharmaceutical composition of Embodiment 14, wherein the aqueous solvent is at least 80% of the pharmaceutical composition by weight.

Embodiment 16 is the pharmaceutical composition of any of Embodiments 1 to 15, wherein the pharmaceutical composition comprises at most 5% organic solvent by weight.

Embodiment 17 is the pharmaceutical composition of any of Embodiments 1 to 16, further comprising an adhesion promoter.

Embodiment 18 is the pharmaceutical composition of any of Embodiments 1 to 17, further comprising a drug release inhibitor.

Embodiment 19 is the pharmaceutical composition of any of Embodiments 1 to 18, further comprising a gel strength modifier.

Embodiment 20 is the pharmaceutical composition of any of Embodiments 1 to 19, further comprising a tackifier.

Embodiment 21 is the pharmaceutical composition of any of Embodiments 1 to 20, wherein upon contact with a mucous membrane, the gelation agent forms a mucoadhesive hydrogel.

Embodiment 22 is the pharmaceutical composition of any of Embodiments 1 to 21, wherein the viscosity of the pharmaceutical composition increases at least 50% after it has been applied to an aqueous boundary.

Embodiment 23 is a container comprising:
the pharmaceutical composition of any of Embodiments 1 to 22; and
an applicator.

Embodiment 24 is a spray container comprising the pharmaceutical composition of any of Embodiments 1 to 22.

Embodiment 25 is an inhaler comprising the pharmaceutical composition of any of Embodiments 1 to 22.

Embodiment 26 is a method of treating an epithelial disorder in a subject in need thereof, the method comprising applying a pharmaceutically active amount of the pharmaceutical composition of any of Embodiments 1 to 22 to a treatment site.

Embodiment 27 is the method of Embodiment 26, wherein the epithelial disorder is a viral or autoimmune disease.

Embodiment 28 is the method of Embodiment 27, wherein the epithelial disorder is a viral disease, and wherein treating the subject results in a decrease of viral gene transcription relative to an untreated subject.

Embodiment 29 is the method of Embodiment 28, wherein the viral gene transcription is decreased by at least 2-fold, at least 5-fold, or at least 10-fold.

Embodiment 30 is the method of any one of Embodiments 26 to 29, wherein the viral disease is a papilloma virus, including human papilloma virus.

Embodiment 31 is the method of Embodiment 26, wherein the epithelial disorder is cancer.

Embodiment 32 is the method of Embodiment 31, wherein treating the subjects results in a decrease in tumor volume relative to an untreated subject.

Embodiment 33 is the method of Embodiment 32, wherein the tumor volume is decreased by at least 5%, at least 10%, at least 50% or at least 90%.

Embodiment 34 is the method of Embodiment 33 wherein the epithelial disorder is human papilloma virus (HPV) infection, eczema, psoriasis, atopic dermatitis, seborrheic dermatitis, histaminergic itch, acne, rosacea, hives, alopecia, shingles, chicken pox, or molluscum contagiosum.

Embodiment 35 is the method of any one of Embodiments 26 to 34, wherein the treatment site is on a mucous membrane or an epidermal surface.

Embodiment 36 is the method of any one of Embodiments 26 to 35, wherein the subject is immunosuppressed.

Embodiment 37 is the method of any one of Embodiments 26 to 36, wherein treating the subject results in a blood level of the protein kinase inhibitor of less than 100 ng/mL.

Embodiment 38 is the method of any one of Embodiments 26 to 37, wherein treating the subject results in decreased phosphorylation of ERK in the treated area relative to an untreated area.

Embodiment 39 is the method of any one of Embodiments 26 to 38, wherein treating the subject results in releasing the active agent at least 1 hour, at least 2 hours, at least 4 hours, at least 12 hours, at least 24 hours, at least 48 hours, or at least 72 hours after application of the pharmaceutical composition.

Embodiment 40 is the method of any one of Embodiments 26 to 39, wherein treating the subject results in reduced incidence of secondary infection relative to an untreated subject.

Embodiment 41 is the method of any one of Embodiments 26 to 40, wherein the subject is at risk of benign tumor formation.

Embodiment 42 is the method of Embodiment 41, wherein treating the subject results in development of a lower volume of benign tumors relative to an untreated subject.

Embodiment 43 is the method of any one of Embodiments 26 to 41, wherein the subject has at least one benign tumor before treatment, and wherein treatment reduces the volume of the benign tumor.

Embodiment 44 is a kit comprising the pharmaceutical composition of any one of Embodiments 1 to 22.

Embodiment 45 is the composition of Embodiment 7 wherein the ERK inhibitor is a p-ERK1/2 inhibitor.

Embodiment 46 is the composition of Embodiment 45, wherein the ERK inhibitor is MK-8353 or a pharmaceutically acceptable salt thereof.

Embodiment 47 is a patch comprising the composition of any one of Embodiments 1 to 22.

Embodiment 48 method of preparing a pharmaceutical composition, the method includes:
preparing a solution comprising combining an inhibitor of a protein kinase or a transcription activator, wherein the protein kinase or transcription activator is a member of a cascade that regulates early viral gene expression or viral genome replication and a gelation agent; and
drying the solution to produce a dried composition.

Embodiment 49 is the method of Embodiment 48, wherein the solution is dried by spray drying or freeze drying.

Embodiment 50 is the method of Embodiment 48 or Embodiment 49, further comprising rehydrating the dried composition.

Embodiment 51 is the method of Embodiment 48 or Embodiment 49, further comprising administering the dried composition to a treatment site.

### EXAMPLES

The present invention is illustrated by the following examples. It is to be understood that the particular examples, materials, amounts, and procedures are to be interpreted broadly in accordance with the scope and spirit of the invention as set forth herein.

TABLE 1

| Reagent | Product name/Number | Source |
|---|---|---|
| 2-(2-ethoxyethoxy)ethanol | Diethylene glycol | Sigma Aldrich |
| | monoethyl ether | |
| Trametinib | | Selleck Chem |
| | | LLC |
| Hydroxypropyl cellulose | Hydroxypropyl | KLUCEL MFX |
| | cellulose, 850 kDa | Pharma |
| HPV16 cell line | SG3 cells maintaining | Paul Lambert, |
| | extrachromosomally | Univ. Wisc. |
| | replicating, circular | |
| | HPV16 genomes | |
| Anti-ß-actin mouse | (ACTN05 (C4)) | Invitrogen |
| monoclonal | MA5-11869 | |
| Secondary sheep anti-mouse | | Fisher Scientific |
| IgG HRP-linked Antibody | | |
| (diluted 1:10,000) | | |
| Phospho-p44/42 MAPK | 4370 | Cell Signaling |
| (Erk1/2) (Thr202/Tyr204) | | |
| (D13.14.4E) XP ® Rabbit | | |
| mAb (diluted 1:1000) | | |
| Secondary Anti-rabbit IgG | 7074 | Cell Signaling |
| HRP-linked Antibody | | |
| (diluted 1:5000) | | |
| Anti-HPV16 E7 Antibody | sc-65711 | Santa Cruz |
| (NM2) (diluted 1:200) | | Biotechnology |
| Hematoxylin & Eosin | Hematoxylin | Epredia |
| Staining | 7231 | |
| | Eosin-Y with | |
| | Phloxine 71304 | |
| Permount | SP15-500 | Fisher Scientific |
| HPV HR7 probe | | Advanced Cell |
| | | Diagnostics |
| ACD DAB | | G Biosciences |
| EcoMount | | BioCare Medical |
| RNase | | Fisher Scientific |
| MK-8353 | SCH900353 | Sellechem |

### Example 1: Topical Delivery of Trametinib Suppresses p-ERK1/2 and HPV E6/E7 mRNA Expression

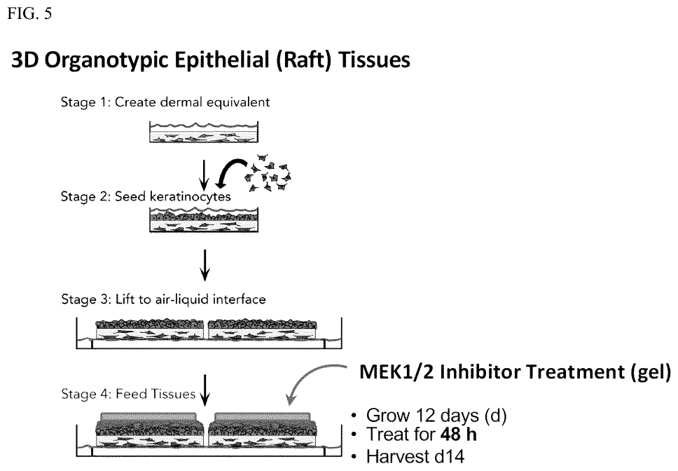

The experimental protocol used in this example is illustrated in FIG. 5. A semi-solid collagen-fibroblast matrix was created to act as a dermal equivalent (stage 1). HPV16-infected SG3 human keratinocytes (cells) that maintained circular HPV16 genomes extrachromosomally were seeded on the top of the dermal equivalent submerged in growth medium (stage 2). After the HPV-infected keratinocytes grew to confluency (2-3 days), growth medium was removed, and the dermal equivalent was lifted to the air-liquid interface above fresh growth medium (stage 3). Fresh medium was replaced every two days. This allowed the keratinocytes to stratify and differentiate into a multilayered epithelium akin to human skin/mucosa within 10-12 days (Stage 4).

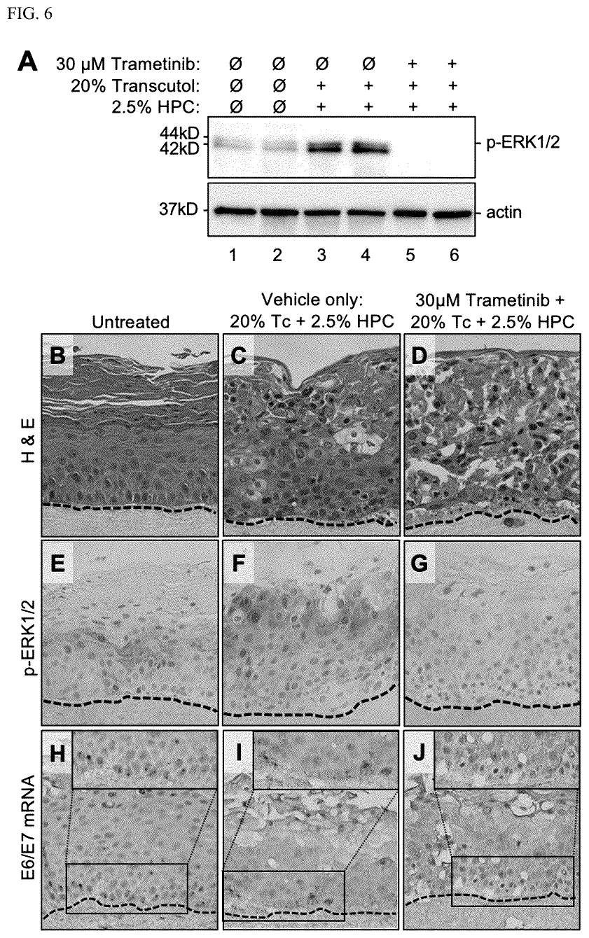

The MEK1/2 inhibitor trametinib was dissolved in diethylene glycol monoethyl ether (TRANSCUTOL; Gattefosse SAS, Saint-Priest, France). The trametinib-2-(2-ethoxyethoxy)ethanol solution was combined with hydroxypropyl cellulose (KLUCEL MXF Pharma, 850 kDa), a pharmaceutical grade thermal gelation agent. Formulations of 30 μM trametinib, 20% 2-(2-ethoxyethoxy)ethanol, and 2.5% hydroxypropyl cellulose applied one time to stratified and differentiated HPV16-infected three-dimensional organotypic epithelial “raft” tissues cultured at the air-liquid interface for 12 days. Each treatment was carried out in duplicate. Tissues were harvested daily two to five days post treatment for total protein extraction and histological analyses (FIG. 6). Specifically, each raft tissue was divided in half with a scalpel. One half was subject to total protein extraction, SDS-PAGE and immunoblot; the second half was fixed in 10% neutral buffered formalin, embedded in paraffin, and sectioned (0.4 μm) on to microscope slides. The tissue sections were subject to histology, immunohistochemistry, or in situ hybridization.

Compared to untreated and vehicle-treated tissue rafts, the topical trametinib-treated tissue rafts showed complete suppression of detectable p-ERK1/2 by immunoblot two days post treatment (FIG. 6A); tissues harvested three to five days post treatment showed this effect was sustained. The same effects were observed by immunohistochemistry for p-ERK1/2 in tissue sections (FIG. 6B-G). In addition, the levels of HPV16 E6/E7 mRNA mirrored those of p-ERK1/2. Specifically, compared to untreated and vehicle-treated tissue rafts, the topical trametinib-treated tissue rafts showed complete suppression of detectable HPV16 E6/E7 mRNAs by in situ hybridization two days post treatment (FIG. 6H-J).

Although the epithelial disruption by the vehicle alone was not ideal (likely due to the high percentage of 2-(2-ethoxyethoxy)ethanol) and seemed to increase p-ERK levels (FIG. 6A lanes 3-4, F), the trametinib in the vehicle completely suppressed p-ERK and HPV activities (FIG. 6A, G, J). Formulations with lower percentages of 2-(2-ethoxyethoxy)ethanol may be prepared to lessen epithelial disruption.

This example shows that one-time topical treatment of HPV-infected tissue with a pharmaceutical composition including 2-(2-ethoxyethoxy)ethanol and trametinib suppressed p-ERK1/2 levels, HPV E6/E7 mRNA transcripts, and hyperplasia.

### Example 2: Formulation of Pharmaceutically Active Compositions Using Varying Percentages of 2-(2-Ethoxyethoxy)Ethanol (TRANSCUTOL, Gattefosse SAS, Saint-Priest, France)

In this example, three additional concentrations of 2-(2-ethoxyethoxy)ethanol were tested to determine whether a formulation with a lower percent 2-(2-ethoxyethoxy)ethanol but an equivalent concentration of trametinib would suppress pERK1/2 expression without the epithelial disruption observed in Example 1. Formulations according to TABLE 2 were prepared. Epithelial raft tissues were prepared as described in Example 1. Each formulation was topically applied to duplicate HPV-infected raft epithelial tissues.

TABLE 2

| | A | B | C | D | E | F | G | H |
|---|---|---|---|---|---|---|---|---|
| HPC (wt %) | 0 | 3.13 | 2.97 | 2.97 | 2.81 | 2.81 | 2.66 | 2.66 |
| 2-(2- | 0 | 0 | 5 | 5 | 10 | 10 | 15 | 15 |
| ethoxyethoxy) | | | | | | | | |
| ethanol (vol %) | | | | | | | | |
| Trametinib | 0 | 0 | 0 | 30 | 0 | 30 | 0 | 30 |
| (μM) | | | | | | | | |
| PBS (vol in ml) | 0 | 100 | 95 | 95 | 90 | 90 | 85 | 85 |

Each formulation was left on the tissue raft undisturbed for 48 hours. 48 hours after treatment, tissues were harvested for protein extraction and histological analysis as described above.

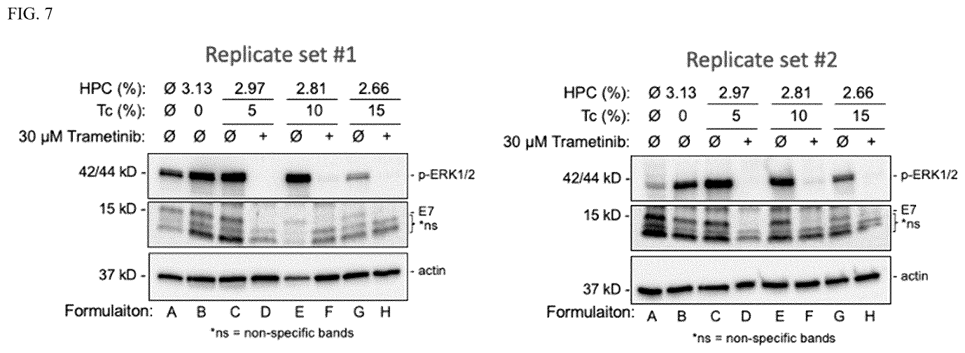

FIG. 7 shows immunoblot data from two replicates of this Example. Lanes correspond to formulations A through H from left to right. As was observed in Example 1, tissues treated with 2-(2-ethoxyethoxy)ethanol lacking trametinib (formulations C, E, G) generally show higher levels of p-ERK1/2 relative to untreated tissues (formulation A). Notably, tissues treated with formulations including 5%, 10%, or 15% 2-(2-ethoxyethoxy)ethanol and trametinib show near complete diminishment of p-ERK1/2 and E7 (FIGS. 7D, F, H). These data indicate that trametinib is effectively delivered to infected cells in formulations including 5%, 10%, or 15% 2-(2-ethoxyethoxy)ethanol. Replicate 2 shows similar results to Replicate 1.

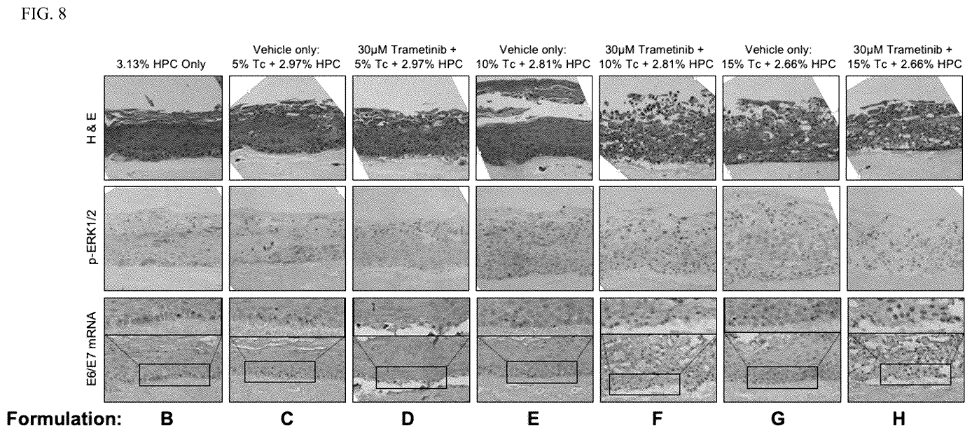

FIG. 8 shows histological images of tissues treated with formulations B through H from left to right (annotated below images). Tissues treated with 15% 2-(2-ethoxyethoxy)ethanol with or without trametinib show physical tissue disruption (Formulations G and H). Tissue treated with 5% or 10% 2-(2-ethoxyethoxy)ethanol without trametinib (Formulations C, E) show minimal tissue disruption and levels of p-ERK1/2 and E6/E7 mRNA like those seen in tissue treated with HPC only (Formulation B). Tissues treated with 5% 2-(2-ethoxyethoxy)ethanol and trametinib (Formulation D) show decreased p-ERK1/2 and E6/E7 mRNA levels relative to tissues treated with a comparable formulation lacking trametinib (Formulation C).

This example shows that formulations with lower weight percentages of 2-(2-ethoxyethoxy)ethanol effectively delivered trametinib with minimal epithelial cell disruption.

### Example 3: Topical Treatment of Epithelial Raft Tissues with Trametinib-Containing Gel-Forming Compositions Suppresses p-ERK1/2 and E7 Protein Levels for Up to Three Days

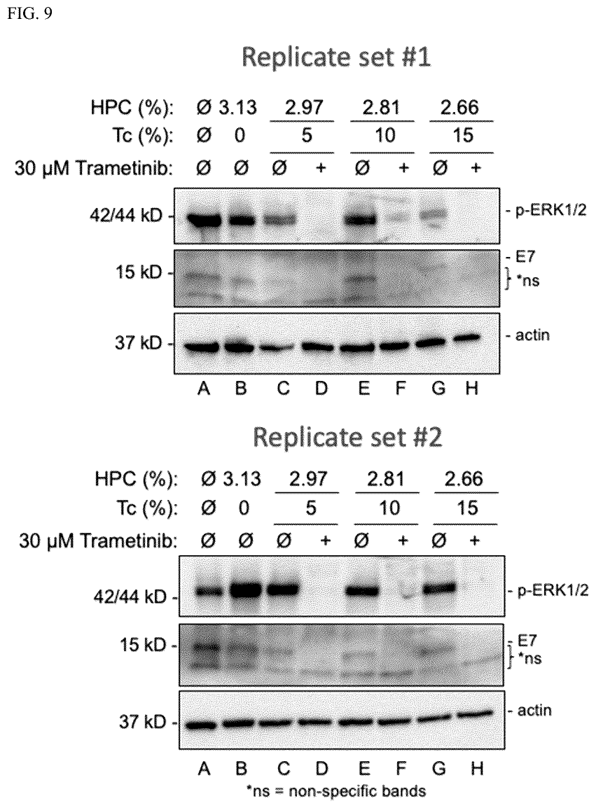

In Example 3, epithelial raft tissues were topically treated in duplicate with formulations including trametinib and 2-(2-ethoxyethoxy)ethanol and analyzed for p-ERK1/2 and E7 protein expression after three days (72 hours). Epithelial raft tissues were prepared as described in Example 1. Formulations were prepared according to TABLE 2. Each epithelial raft tissue was topically treated with one formulation. 72 hours after treatment, epithelial raft tissues were collected for protein extraction. FIG. 9 shows immunoblotting data from two replicates of this protocol. Lanes correspond to protein extracted from tissue treated with formulations A through H from left to right. Treatment with 5%, 10%, or 15% 2-(2-ethoxyethoxy)ethanol and trametinib appeared to decrease both p-ERK1/2 and HPV16 E7 levels. Replicate 2 shows trends similar to those observed in Replicate 1.

This example shows that one-time topical treatment of epithelial raft tissues suppresses cellular p-ERK1/2 signaling and markers of HPV activity for at least 72 hours.

### Example 4: Topical Treatment of Epithelial Raft Tissues with Trametinib-Containing Gel-Forming Compositions Suppresses p-ERK1/2 and E7 Protein Levels for Up to Four Days

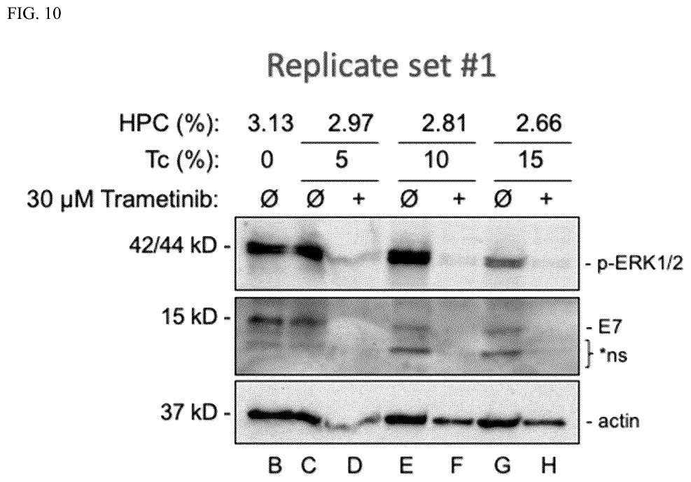

In Example 4, epithelial raft tissues were topically treated with formulations including trametinib and 2-(2-ethoxyethoxy)ethanol and analyzed for p-ERK1/2 and E7 protein expression after four days (96 hours). Epithelial raft tissues were prepared as described in Example 1. Formulations were prepared according to TABLE 2. Each epithelial raft tissue was topically treated with one formulation. 96 hours after treatment, epithelial raft tissues were collected for protein extraction. FIG. 10 shows immunoblotting data of protein extracted from tissue treated with formulations B through H. For this example, no epithelial tissue raft was treated with formulation A. As was observed in EXAMPLE 2 and EXAMPLE 3, treatment with trametinib, but not treatment with 5% or 10% 2-(2-ethoxyethoxy)ethanol alone appears to decrease p-ERK1/2 and E7 levels. In particular, lanes showing proteins from tissue treated with formulations including 10% 2-(2-ethoxyethoxy)ethanol with or without trametinib in particular shows a decrease in p-ERK1/2 and E7 levels.

This example shows that one-time topical treatment of epithelial raft tissues suppresses markers of viral activity for at least 96 hours.

### Example 5. Topical Delivery of MK-8353 in 2-(2-Ethoxyethoxy)Ethanol Suppresses p-ERK1/2 Activity as Measured by p-Fra1 Phosphorylation

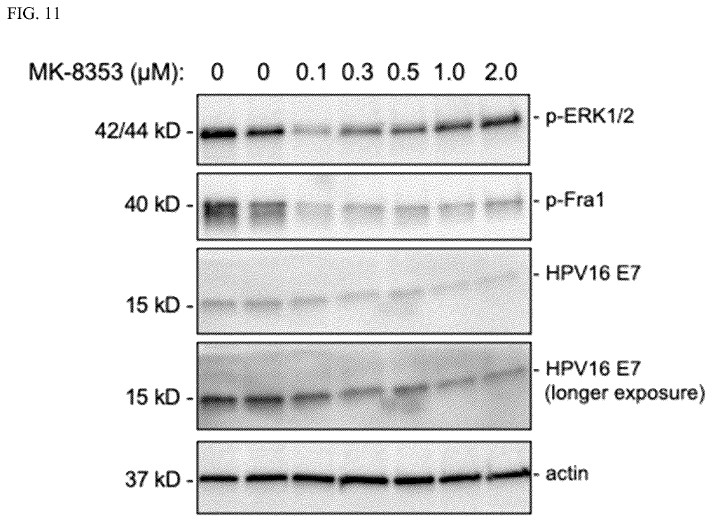

The p-ERK1/2 inhibitor MK-8353 (Selleck Chemicals LLC, Houston, TX) was dissolved in 2-(2-ethoxyethoxy)ethanol (Tc) at 5 mM. HPV16-infected SG3 cells were plated in complete medium and then treatments were initiated without active agent or with increasing concentrations of MK-8353 in serum-free medium. At 24 h post treatment, fresh treatments were added and maintained for an additional 24 h. Cells treated for 48 h were lysed with detergent-containing buffer and 50 μg of each sample was analyzed by SDS-PAGE and immunoblot for the proteins of interest including p-ERK1/2, p-Fra1, HPV16 E7, and actin. This immunoblot is shown in FIG. 11.

While the p-ERK1/2 inhibitor does not necessarily prevent the phosphorylation of ERK1/2 (p-ERK1/2), it does inhibit the activity of p-ERK1/2 by preventing the phosphorylation of the downstream effector, p-Fra1. Preventing the activity of p-ERK/2 (and Fra-1) also leads to a decrease in the levels of the HPV16 E7 oncoprotein.

These data show that MK-8353 can be dissolved in 2-(2-ethoxyethoxy)ethanol (as opposed to DMSO), which does not alter the active agent's ability to inhibit the activity of p-ERK1/2 and subsequently reduce the levels of the HPV16 E7 oncoprotein.

### Example 6. Thermally Gelling Spray Compositions

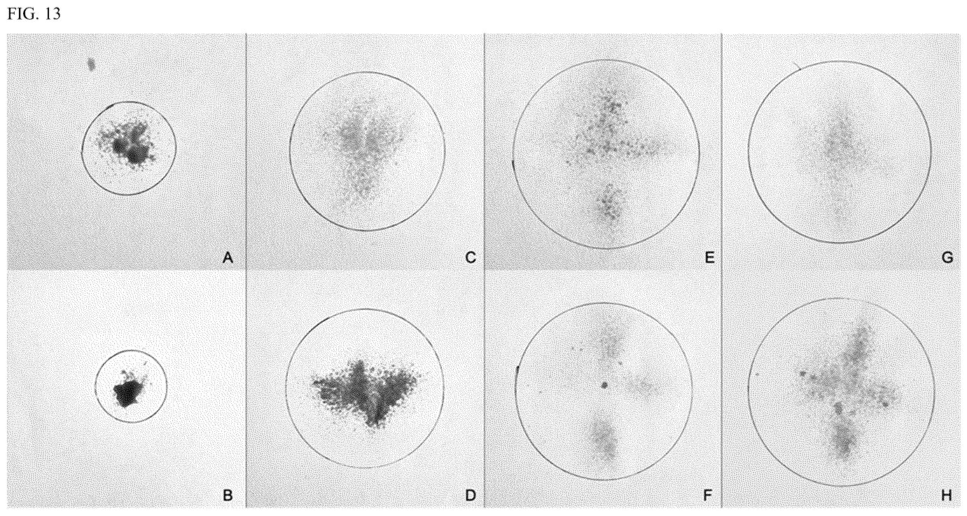

The following compositions were prepared as base formulations and can contain an active MEKi or ERKi in a therapeutically effective amount. Hydroxypropyl methylcellulose (HPMC; 2910 substitution type) solutions at varying concentrations and molecular weights (METHOCEL E6 or METHOCEL E50, The Dow Chemical Company, Midland, MI) and containing blue food coloring were tested using a high viscosity spray bottle positioned at a standardized USP aerosol cascade impaction induction port distance of 7.9 cm (standard distance obtained from a USP Apparatus 7 induction port) from a white paper target. A single spray atomization was actuated, and the resulting features of the spray dispersion radius and impact droplet size were evaluated by circling the outermost observed spray droplets from the center of impact, and by visual inspection of droplets. Each concentration was additionally tested with the inclusion of the potent absorption enhancer diethylene glycol monoethyl ether (DGME) at 15% w/v to investigate its effect on the spray pattern properties (FIG. 13).

### Example 7. Rheological Sol-Gel Transition Temperatures

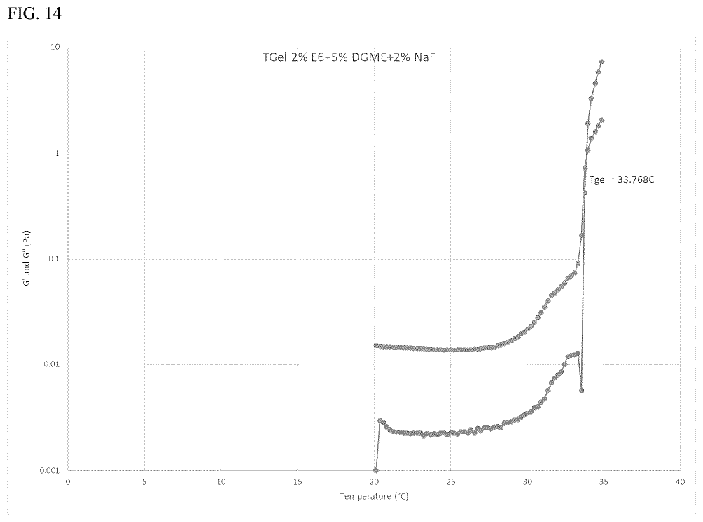

The following compositions were prepared as base formulations and can contain an active MEKi or ERKi in a therapeutically effective amount. Spray Sol-Gel transition evaluation was performed with 1.5 or 2% w/v sodium fluoride (NaF) concentrations with the 2% w/v METHOCEL (The Dow Chemical Company, Midland, MI) E6+DGME. Thermal gelation temperature below body temperature at approximately 34° C. with a 2% w/v NaF containing formulation is observed (FIG. 14).

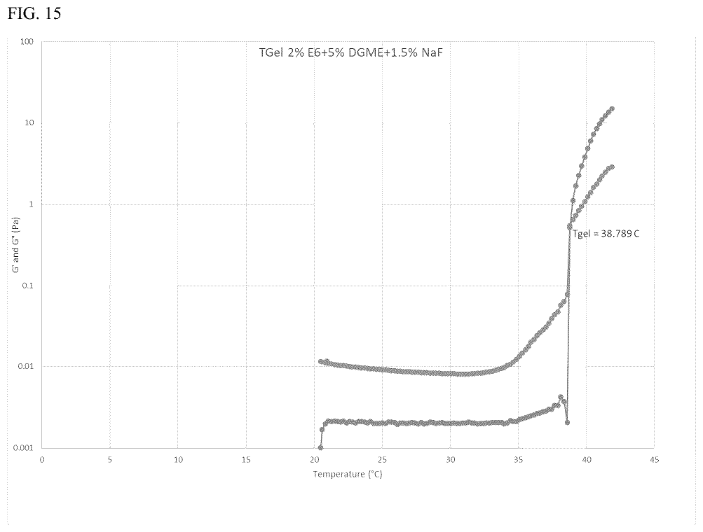

Thermal gelation temperature above body temperature at approximately 39° C. with a 1.5% NaF containing formulation is observed (FIG. 15).

As indicated, the Tgel for the spray composition can be effectively controlled by adjusting the amount of gelling aid. For one or more applications, it may be desirable to demonstrate Tgel at or below body temperature.

### Example 8. Spray Sol-Gel Transition

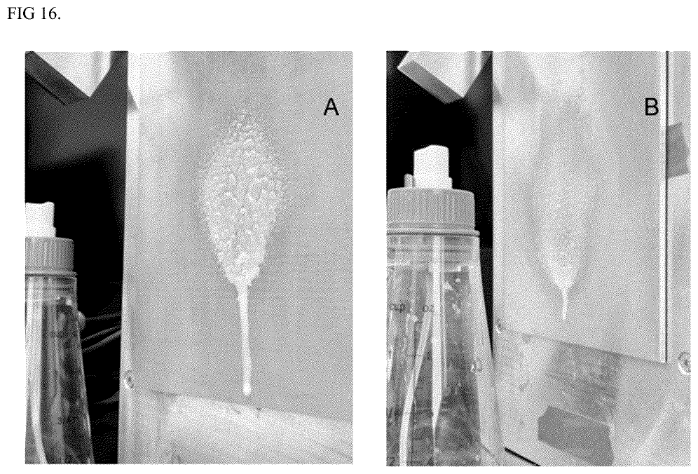

The following compositions were prepared as base formulations and can contain an active MEKi or ERKi in a therapeutically effective amount. Spray Sol-Gel transition evaluation was performed with a 1.5 or 2% w/v NaF sodium fluoride concentrations with the 2% w/v METHOCEL (The Dow Chemical Company, Midland, MI) E6+DGME by spraying onto a 37° C. hot plate at a distance of 7.9 cm (standard distance obtained from a USP Apparatus 7 induction port) using a high-viscosity spray bottle. Thermal gelation was not observed for the 1.5% w/v NaF solution upon contact with the heated surface; the liquid formulation was seen to creep/flow down the target and did not solidify until evaporation had occurred (FIG. 16). Conversely, the 2.0% w/v NaF solution gelled rapidly; very little liquid creep was seen before Sol-Gel transition. These observations corroborate the rheological findings related to the loss/viscous modulus (G″) and the elastic/storage modulus (G′) in Example 7 above.

### Example 9. Rheological Sol-Gel Transition Temperature of an Isotonic Composition

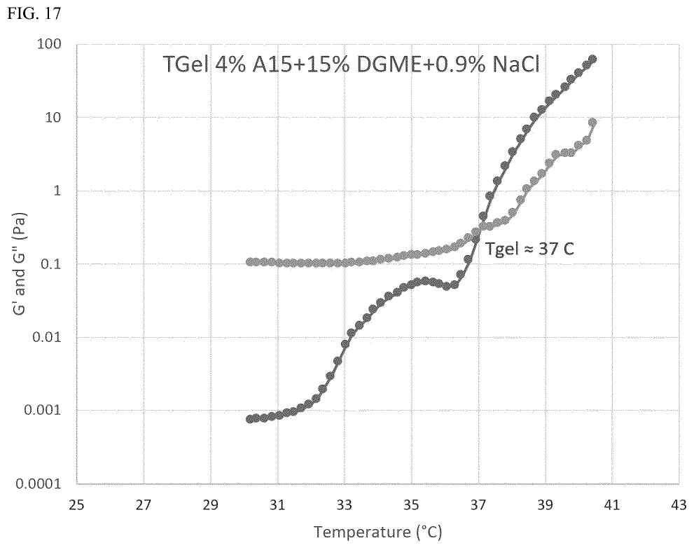

The following composition was prepared as base formulation and can contain an active MEKi or ERKi in a therapeutically effective amount. Sol-Gel transition evaluation was performed with a 0.9% w/v sodium chloride (NaCl) concentration with the 2% w/v METHOCEL (The Dow Chemical Company, Midland, MI) A15+15% DGME. FIG. 17 shows a thermal gelation temperature at approximately body temperature of 37° C. with the isotonic formulation.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

As used herein, “have,” “has,” “having,” “include,” “includes,” “including,” “comprise,” “comprises,” “comprising” or the like are used in their open-ended inclusive sense, and generally mean “include, but not limited to,” “includes, but not limited to,” or “including, but not limited to.” Further, wherever embodiments are described herein with the language “have,” “has,” “having,” “include,” “includes,” “including,” “comprise,” “comprises,” “comprising” and the like, otherwise analogous embodiments described in terms of “consisting of” and/or “consisting essentially of” are also provided. The term “consisting of” means including, and limited to, that which follows the phrase “consisting of” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A pharmaceutical composition comprising:
an inhibitor of a protein kinase or an inhibitor of a transcription activator, wherein the protein kinase or transcription activator is a member of a cascade that regulates early viral gene expression or viral genome replication;
a gelation agent; and
an aqueous solvent.

2. The pharmaceutical composition of claim 1, wherein the gelation agent comprises hydroxypropyl methylcellulose, methylcellulose, or hydroxypropyl cellulose.

3. The pharmaceutical composition of claim 1, wherein the gelation agent has a molecular weight of 10 kDa to 2,000 kDa.

4. The pharmaceutical composition of claim 1, wherein the protein kinase inhibitor is an MEK inhibitor or an ERK inhibitor.

5. The pharmaceutical composition of claim 4, wherein the MEK inhibitor is trametinib or cobimetinib, or a pharmaceutically acceptable salt thereof.

6. The pharmaceutical composition of claim 4, wherein the ERK inhibitor is MK-8353 or a pharmaceutically acceptable salt thereof.

7. The pharmaceutical composition of claim 1, wherein the aqueous solvent comprises water, alcohol, or a combination thereof.

8. The pharmaceutical composition of claim 7, wherein the alcohol comprises 2-(2-ethoxyethoxy)ethanol.

9. The pharmaceutical composition of claim 1, wherein the aqueous solvent is at least 40% of the pharmaceutical composition by weight.

10. The pharmaceutical composition of claim 9, wherein the aqueous solvent is at least 80% of the pharmaceutical composition by weight.

11. The pharmaceutical composition of claim 1, wherein the pharmaceutical composition comprises at most 5% organic solvent by weight.

12. The pharmaceutical composition of claim 1, wherein the gelation agent forms a mucoadhesive hydrogel upon contact with a mucous membrane.

13. The pharmaceutical composition of claim 12, wherein viscosity of the pharmaceutical composition increases at least 50% after it has been applied to an aqueous boundary.

14. The pharmaceutical composition of claim 1, wherein the composition forms a gel before it is applied to a treatment site.

15. A active agent delivery device comprising the pharmaceutical composition of claim 1.

16. The active agent delivery device of claim 15, wherein the active agent delivery device comprises a transdermal active agent delivery device or topical active agent delivery device.

17. The active agent delivery device of claim 15, wherein the active agent delivery device comprises a bandage, a wrap, a plaster, or a dressing.

18. A container comprising:
the pharmaceutical composition of claim 1; and
an applicator.

19. A kit comprising the pharmaceutical composition of claim 1.

20. A method of treating an epithelial disorder in a subject, the method comprising applying a pharmaceutically active amount of the pharmaceutical composition of claim 1 to a treatment site.

21. The method of claim 20, wherein the epithelial disorder is a viral disease, and wherein treating the subject results in a decrease of viral gene transcription relative to an untreated subject.

22. The method of claim 21, wherein the viral disease is infection by a papilloma virus.

23. The method of claim 20, wherein the epithelial disorder is cancer, and wherein treating the subjects results in a decrease in tumor volume relative to an untreated subject.

24. The method of claim 20, wherein the treatment site is on a mucous membrane or an epidermal site.

25. The method of claim 20, wherein treating the subject results in a blood level of the protein kinase inhibitor of less than 100 ng/mL.

26. The method of claim 20, wherein treating the subject results in active agent release at least 72 hours after application of the pharmaceutical composition.

27. A method of preparing a pharmaceutical composition, the method comprising:
preparing a solution comprising combining an inhibitor of a protein kinase or a transcription activator, wherein the protein kinase or transcription activator is a member of a cascade that regulates early viral gene expression or viral genome replication and a gelation agent; and
drying the solution to produce a dried composition.

28. The method of claim 27, wherein the solution is dried by spray drying or freeze drying.

29. The method of claim 27, further comprising rehydrating the dried composition.

30. The method of claim 27, further comprising administering the dried composition to a treatment site.
